(19) United States
(12) Patent Application Publication
(10) Pub. No.: US 2025/0152911 A1
(43) Pub. Date: May 15, 2025

(54) AWAKENING INDUCING DEVICE, AWAKENING INDUCING METHOD, AND RECORDING MEDIUM

(71) Applicant: Panasonic Automotive Systems Co., Ltd., Kanagawa (JP)

(72) Inventor: Kazuhiko MAEDA, Osaka (JP)

(73) Assignee: Panasonic Automotive Systems Co., Ltd., Kanagawa (JP)

(21) Appl. No.: 19/024,542

(22) Filed: Jan. 16, 2025

Related U.S. Application Data
(63) Continuation of application No. PCT/JP2023/022586, filed on Jun. 19, 2023.

(30) Foreign Application Priority Data
Sep. 16, 2022 (JP) 2022-147602

(51) Int. Cl. A61M 21/00

(52) U.S. Cl. CPC A61M 21/00; A61M 2021/0022; A61M 2021/0027; A61M 2021/0044; A61M 2021/0083

(57) ABSTRACT

An awakening inducing device includes: a first awakener that induces awakening of a user by periodically repeating a first stimulation period in which a stimulus of a first type is applied to the user; a second awakener that induces awakening of the user by periodically repeating a second stimulation period in which a stimulus of a second type is applied to the user; and a controller that causes the first awakener and the second awakener to have a synchronization cycle and an offset cycle, the synchronization cycle being a cycle in which a start timing of the first stimulation period and a start timing of the second stimulation period are synchronized with each other, the offset cycle being a cycle in which the start timing of the first stimulation period and the start timing of the second stimulation period are offset from each other.

### CROSS REFERENCE TO RELATED APPLICATIONS

This is a continuation application of PCT International Patent Application No. PCT/JP2023/022586 filed on Jun. 19, 2023, designating the United States of America, which is based on and claims priority of Japanese Patent Application No. 2022-147602 filed on Sep. 16, 2022.

### FIELD

The present disclosure relates to an awakening inducing device, an awakening inducing method, a recording medium.

### BACKGROUND

Conventionally, there are devices that awaken a user by giving vibration stimulation to a specific area (portion) on the user (see, for example, Patent Literature (PTL) 1).

PTL 1 discloses a device that awakens a user effectively by giving vibration stimulation to the tendon portion of the user's latissimus dorsi muscle or the tendon portion of the user's gluteus medius muscle.

### CITATION LIST

### Patent Literature

PTL 1: Japanese Unexamined Patent Application Publication No. 2016-153969

### Non Patent Literature

NPL 1: Audio-tactile superiority over visuo-tactile and audio-visual combinations in the temporal resolution of synchrony perception, Waka Fujisaki & Shinya Nishida, Experimental Brain Research volume 198, pages 245-259 (2009).

### SUMMARY

The technique of devices that awaken users can be improved upon.

In view of this, the present disclosure provides an awakening inducing device and so on capable of improving upon the above related art.

In order to address the above, an awakening inducing device according to an aspect of the present disclosure includes: a first awakener that induces awakening of a user by periodically repeating a first stimulation period in which a stimulus of a first type is applied to the user and a first suspension period in which no stimulus is applied to the user; a second awakener that induces awakening of the user by periodically repeating a second stimulation period in which a stimulus of a second type is applied to the user and a second suspension period in which no stimulus is applied to the user; and a controller that causes the first awakener and the second awakener to have a synchronization cycle and an offset cycle, the synchronization cycle being a cycle in which a start timing of the first stimulation period and a start timing of the second stimulation period are synchronized with each other, the offset cycle being a cycle in which the start timing of the first stimulation period and the start timing of the second stimulation period are offset from each other.

An awakening inducing method according to another aspect of the present disclosure includes: inducing awakening of a user by periodically repeating a first stimulation period in which a stimulus of a first type is applied to the user and a first suspension period in which no stimulus is applied to the user; inducing awakening of the user by periodically repeating a second stimulation period in which a stimulus of a second type is applied to the user and a second suspension period in which no stimulus is applied to the user; and causing the stimulus of the first type and the stimulus of the second type applied to the user to have a synchronization cycle and an offset cycle, the synchronization cycle being a cycle in which a start timing of the first stimulation period and a start timing of the second stimulation period are synchronized with each other, the offset cycle being a cycle in which the start timing of the first stimulation period and the start timing of the second stimulation period are offset from each other.

A recording medium according to still another aspect of the present disclosure is a non-transitory computer-readable recording medium for use in a computer. The recording medium has recorded thereon a computer program for causing the computer to execute the above-described awakening inducing method.

The awakening inducing device and so on according to some aspects of the present disclosure can be improved upon.

### BRIEF DESCRIPTION OF DRAWINGS

These and other advantages and features of the present disclosure will become apparent from the following description thereof taken in conjunction with the accompanying drawings that illustrate a specific embodiment of the present disclosure.

FIG. 1 is a side view illustrating an awakening inducing system according to Embodiment 1.

FIG. 2 is a top view for explaining a positional relationship between vibration devices according to Embodiment 1 and a user.

FIG. 3 is a block diagram illustrating a functional configuration of the awakening inducing system according to Embodiment 1.

FIG. 4 is an illustration for explaining a position on a user to which the vibration devices according to Embodiment 1 give vibration stimulation.

FIG. 5 is a diagram illustrating an example of an eccentric motor.

FIG. 6A is a flowchart for explaining a process executed by the awakening inducing system according to Embodiment 1.

FIG. 6B is a flowchart for explaining a part of the process executed by the awakening inducing system according to Embodiment 1.

FIG. 6C is a graph for explaining an appropriate vibration period of the awakening inducing system according to Embodiment 1.

FIG. 6D is a diagram for explaining the relationship between a first cycle and a second cycle of the awakening inducing system according to Embodiment 1.

FIG. 7A is a graph for explaining effects of the awakening inducing system according to Embodiment 1.

FIG. 7B is a graph for explaining, in more detail, the effect of the awakening inducing system according to Embodiment 1.

FIG. 8 is a top view illustrating an awakening inducing system according to Embodiment 2.

FIG. 9 is a diagram for explaining a positional relationship between a vibration device according to Embodiment 2 and a user.

FIG. 10 is a block diagram illustrating a functional configuration of the awakening inducing system according to Embodiment 2.

FIG. 11 is a flowchart for explaining a process executed by the awakening inducing system according to Embodiment 2.

FIG. 12 is a block diagram illustrating a functional configuration of an awakening inducing system according to Embodiment 3.

FIG. 13 is an illustration for explaining a position on a user to which a vibration device according to Embodiment 3 gives vibration stimulation.

FIG. 14 is a diagram illustrating an example of a pressure distribution across a seating surface of a seat.

FIG. 15 is a flowchart for explaining a process executed by the awakening inducing system according to Embodiment 3.

FIG. 16 is a diagram illustrating a first variation of a vibration device.

FIG. 17 is a diagram illustrating a second variation of a vibration device.

FIG. 18 is a diagram illustrating a variation in how vibration devices are arranged.

### DESCRIPTION OF EMBODIMENTS

### Overview of the Present Disclosure

The inventors discovered the following problems related to the related arts described above. In other words, devices that induce awakening of a user are desired to be improved upon more appropriately performing the inducing. More specifically, to increase the drowsiness eliminating effect, that is, to awaken a user fully, the nerve activity level of the supplementary motor area of the user may be increased. The supplementary motor area is known not to be activated by vibration stimulation that causes no motion illusion but to be activated by vibration stimulation that causes a motion illusion. In other words, giving a user vibration stimulation that causes a clear motion illusion can activate the supplementary motor area of the user, that is, can increase the nerve activity level of the supplementary motor area of the user. Furthermore, activating the supplementary motor area can lead to increased stimulation to, for example, the reticular formation (the brainstem reticular formation). Stimulating the reticular formation awakens the user. In other words, giving a user vibration stimulation that causes a clear motion illusion can awaken the user effectively.

Through diligent study, the inventors of the present application have found that giving vibration stimulation to a specific area on a user intermittently on a predetermined vibration condition can produce a more intense illusion (motion illusion). In other words, the inventors have found that giving vibration stimulation to a specific area on a user makes it possible to awaken the user more fully than conventional techniques. When such an intermittent vibration stimulus is applied suddenly from a state where the vibration stimulus is not applied, the user may be surprised, which may have an adverse effect on work performed thus far. Hence, the present disclosure will also mention reducing a surprise to a user when the vibration stimulus is applied, by playing back a musical piece in a playback mode to allow the user to recognize beforehand that the vibration may be applied to the user soon.

On the other hand, the reduction of a surprise in such a configuration to play back the musical piece and apply the vibration stimulus, that is, two stimuli, leads to a decrease in the effect of awakening the user. Hence, the present disclosure will also mention making it difficult to decrease the effect of awakening the user while reducing a surprise to the user by causing the user to feel a sense of freshness when the stimuli are applied. In particular, the effect produced by this sense of freshness acts irrespective of the types of the two stimuli and is thus effective also in a combination of types of stimuli other than the combination of the playback of the musical piece and the vibration stimulus. Furthermore, as the types of stimuli, stimuli of the same type may be combined. That is, a stimulus of a first type and a stimulus of a second type combined here may be the same type.

An awakening inducing device according to a first aspect of the present disclosure includes: a first awakener that induces awakening of a user by periodically repeating a first stimulation period in which a stimulus of a first type is applied to the user and a first suspension period in which no stimulus is applied to the user; a second awakener that induces awakening of the user by periodically repeating a second stimulation period in which a stimulus of a second type is applied to the user and a second suspension period in which no stimulus is applied to the user; and a controller that causes the first awakener and the second awakener to have a synchronization cycle and an offset cycle, the synchronization cycle being a cycle in which a start timing of the first stimulation period and a start timing of the second stimulation period are synchronized with each other, the offset cycle being a cycle in which the start timing of the first stimulation period and the start timing of the second stimulation period are offset from each other.

In the above awakening inducing device, the stimulus of the first type and the stimulus of the second type are applied to the user such that, in the offset cycle, their stimulation periods (first stimulation period and the second stimulation period) are offset to start from different time points, while in the offset cycle, their stimulation periods start in synchronization with each other. Therefore, in the offset cycle, the offset can cause the sense of freshness in the user. The sense of freshness can increase the effects of inducing awakening of the user by giving the stimulation. Therefore, it is possible to prevent the decrease of the effects even if the user gets used to stimulation of two types of stimuli given in synchronization with each other, guessing a start timing of a stimulation period of one type of stimulus from the other type of stimulus. As a result, the awakening inducing device can more appropriately induce awakening of the user than the related arts.

The awakening inducing device according to a second aspect of the present disclosure is the awakening inducing device according to the first aspect of the present disclosure, wherein the stimulus of the first type is sound, and the stimulus of the second type is vibration.

With this, the use of the stimulus that is sound and the stimulus that is vibration can more appropriately induce awakening of the user than the related arts.

The awakening inducing device according to a third aspect of the present disclosure is the awakening inducing device according to the first aspect of the present disclosure, wherein the stimulus of the first type is light, and the stimulus of the second type is sound.

With this, the use of the stimulus that is light and the stimulus that is sound can more appropriately induce awakening of the user than the related arts.

The awakening inducing device according to a fourth aspect of the present disclosure is the awakening inducing device according to the first aspect of the present disclosure, wherein the stimulus of the first type is light, and the stimulus of the second type is vibration.

With this, the use of the stimulus that is light and the stimulus that is vibration can more appropriately induce awakening of the user than the related arts.

The awakening inducing device according to a fifth aspect of the present disclosure is the awakening inducing device according to first aspect of the present disclosure, wherein the stimulus of the first type is vibration, and the stimulus of the second type is vibration different from the vibration of the stimulus of the first type.

With this, the stimulation of two different types of vibration can more appropriately induce awakening of the user than the related arts.

The awakening inducing device according to a sixth aspect of the present disclosure is the awakening inducing device according to the second aspect of the present disclosure, wherein a length of the offset between the start timing of the first stimulation period and the start timing of the second stimulation period is at least 40 milliseconds (ms) and at most half of the second suspension period.

With this, it is possible to more easily ensure a time resolution that enables a human to perceive the offset between the stimulation of sound and the stimulation of vibration. As a result, it is possible to set an appropriate amount of the offset to reduce interference of a stimulation period with a next stimulation period.

The awakening inducing device according to a seventh aspect of the present disclosure is the awakening inducing device according to any one of the first to sixth aspects of the present disclosure, wherein the controller causes the first awakener and the second awakener to repeat a cycle pattern that includes the offset cycle once and the synchronization cycle one or more times in a predetermined order.

With this, the stimulation of repeating a cycle pattern that includes one offset cycle and one or more synchronization cycles in a predetermined order can more appropriately induce awakening of the user than the related arts.

The awakening inducing device according to an eighth aspect of the present disclosure is the awakening inducing device according to any one of the first to sixth aspects of the present disclosure, wherein the controller causes the first awakener and the second awakener to repeat a cycle pattern that includes the offset cycle once and the synchronization cycle one or more times, and the offset cycle and the synchronization cycle are in a random order in the cycle pattern.

With this, the stimulation of repeating a cycle pattern that includes one offset cycle and one or more synchronization cycles in a random order can more appropriately induce awakening of the user than the related arts.

The awakening inducing device according to a ninth aspect of the present disclosure is the awakening inducing device according to any one of the first to eighth aspects of the present disclosure that further includes: a drowsiness estimator that estimates a state of drowsiness of the user, wherein the controller causes the first awakener and the second awakener to have the synchronization cycle only, when the state of drowsiness estimated is closer to an awake state than a threshold state is, and the controller causes the first awakener and the second awakener to have both the synchronization cycle and the offset cycle, when the state of drowsiness estimated is closer to an asleep state than the threshold state is.

With this, when the state of drowsiness of the user is closer to an asleep state than the threshold state is, stimulation including the synchronization cycle and the offset cycle can more appropriately induce awakening of the user than the related arts.

The awakening inducing device according to a tenth aspect of the present disclosure is the awakening inducing device according to any one of the first to ninth aspects of the present disclosure that further includes: a drowsiness estimator that estimates a state of drowsiness of the user, wherein the controller changes, based on the state of drowsiness estimated, a stimulation condition for stimulation applied to the user by at least one of the first awakener or the second awakener.

With this, stimulation on a condition corresponding to the state of drowsiness of the user can more appropriately induce awakening of the user than the related arts.

The awakening inducing device according to an eleventh aspect of the present disclosure is the awakening inducing device according to any one of the first to tenth aspects of the present disclosure, wherein the drowsiness estimator estimates a level of drowsiness of the user as the state of drowsiness, and the controller changes the stimulation condition for stimulation applied to the user by at least one of the first awakener or the second awakener, when the level of drowsiness estimated exceeds a predetermined level.

With this, stimulation on a condition corresponding to the level of drowsiness of the user can more appropriately induce awakening of the user than the related arts.

The awakening inducing device according to a twelfth aspect of the present disclosure is the awakening inducing device according to the tenth aspect of the present disclosure, wherein the drowsiness estimator estimates a temporal change of a level of drowsiness of the user as the state of drowsiness, and the controller changes the stimulation condition for stimulation applied to the user by at least one of the first awakener or the second awakener, when a degree of the temporal change estimated is equal to or greater than a predetermined degree.

With this, stimulation on a condition corresponding to a degree of a temporal change of the level of drowsiness of the user can more appropriately induce awakening of the user than the related arts.

The awakening inducing device according to a thirteenth aspect of the present disclosure is the awakening inducing device according to the tenth aspect of the present disclosure, wherein the drowsiness estimator estimates a future level of drowsiness of the user as the state of drowsiness, and the controller changes the stimulation condition for stimulation applied to the user by at least one of the first awakener or the second awakener, when the future level of drowsiness estimated exceeds a predetermined level.

With this, stimulation on a condition corresponding to a level of drowsiness of the user in the future can more appropriately induce awakening of the user than the related arts.

The awakening inducing device according to a fourteenth aspect of the present disclosure is the awakening inducing device according to any one of the first to ninth aspects of the present disclosure, wherein the controller randomly changes a stimulation condition for stimulation applied to the user by at least one of the first awakener or the second awakener.

With this, stimulation under a random condition can more appropriately induce awakening of the user than the related arts.

The awakening inducing device according to a fifteenth aspect of the present disclosure is the awakening inducing device according to any one of the tenth to fourteenth aspects of the present disclosure, wherein the controller changes, as the stimulation condition, a frequency of the offset cycle with respect to the synchronization cycle.

With this, stimulation in which a frequency of the offset cycles with respect to the synchronization cycles varies can more appropriately induce awakening of the user than the related arts.

The awakening inducing device according to a sixteenth aspect of the present disclosure is the awakening inducing device according to any one of the tenth to fourteenth aspects of the present disclosure, wherein the controller changes, as the stimulation condition, a length of the offset between the start timing of the first stimulation period and the start timing of the second stimulation period in the offset cycle.

With this, stimulation in which the length of offset between the start timing of the first stimulation period and the start timing of the second stimulation period in the offset cycle varies can more appropriately induce awakening of the user than the related arts.

The awakening inducing device according to a seventeenth aspect of the present disclosure is the awakening inducing device according to any one of the tenth to fourteenth aspects of the present disclosure, wherein at least one of the stimulus of the first type or the stimulus of the second type is vibration, and the controller changes, as the stimulation condition, a vibration frequency of the vibration applied by at least one of the first awakener or the second awakener.

With this, stimulation in which the vibration frequency of vibration stimulation applied by at least one of the first awakener or the second awakener varies can more appropriately induce awakening of the user than the related arts.

The awakening inducing device according to an eighteenth aspect of the present disclosure is the awakening inducing device according to any one of the tenth to fourteenth aspects of the present disclosure, wherein the controller changes, as the stimulation condition, a length of at least one of the stimulus of the first type or the stimulus of the second type.

With this, stimulation in which the length of at least one of the stimulus of the first type or the stimulus of the second type varies can more appropriately induce awakening of the user than the related arts.

The awakening inducing device according to a nineteenth aspect of the present disclosure is the awakening inducing device according to any one of the tenth to fourteenth aspects of the present disclosure, wherein at least one of the stimulus of the first type or the stimulus of the second type is vibration, at least one of the first awakener or the second awakener applies the vibration to two or more different portions on the user, and the controller changes, as the stimulation condition, the two or more different portions on which the vibration is applied by the at least one of the first awakener or the second awakener to two or more other portions on the user.

With this, stimulation in which a portion to be applied with vibration by at least one of the first awakener or the second awakener varies can more appropriately induce awakening of the user than the related arts.

The awakening inducing device according to a twentieth aspect of the present disclosure is the awakening inducing device according to any one of the tenth to fourteenth aspects of the present disclosure, wherein the controller changes, as the stimulation condition, an intensity of at least one of the stimulus of the first type or the stimulus of the second type.

With this, stimulation in which the intensity of at least one of the stimulus of the first type or the stimulus of the second type varies can more appropriately induce awakening of the user than the related arts.

An awakening inducing method according to a twenty-first aspect of the present disclosure includes: inducing awakening of a user by periodically repeating a first stimulation period in which a stimulus of a first type is applied to the user and a first suspension period in which no stimulus is applied to the user; inducing awakening of the user by periodically repeating a second stimulation period in which a stimulus of a second type is applied to the user and a second suspension period in which no stimulus is applied to the user; and causing the stimulus of the first type and the stimulus of the second type applied to the user to have a synchronization cycle and an offset cycle, the synchronization cycle being a cycle in which a start timing of the first stimulation period and a start timing of the second stimulation period are synchronized with each other, the offset cycle being a cycle in which the start timing of the first stimulation period and the start timing of the second stimulation period are offset from each other.

The above awakening inducing device can produce the same effects as those produced by the previously-described awakening inducing device.

A recording medium according to a twenty-second aspect of the present disclosure is a non-transitory computer-readable recording medium for use in a computer, the recording medium having recorded thereon a program for causing the computer to execute: the awakening inducing method described above.

The above recording medium can produce, by using the computer, the same effects as those produced by the previously-described awakening inducing method.

Hereinafter, some embodiments of the present disclosure will be described with reference to the drawings. The embodiments described below merely illustrate general or specific examples of the present disclosure. Hence, the numerical values, the constituent elements, the arrangement positions and the connection modes of the constituent elements, the processes (the steps), the order of the processes, and so on illustrated in the following embodiments are examples and are not intended to limit the present disclosure.

Moreover, the drawings are schematic diagrams and do not necessarily provide the exact depictions. Hence, the scales and so on do not necessarily match among the drawings. In the drawings, substantially identical components are given identical reference characters, and duplicate descriptions thereof may be omitted or simplified.

In the present specification and the drawings, the X-axis, the Y-axis, and the Z-axis represent the three axes of a three-dimensional orthogonal coordinate system. In the following embodiments, the Z-axis direction is the vertical direction, and the direction perpendicular to the Z-axis (the direction parallel to the XY-plane) is the horizontal direction. In addition, the positive direction along the Z-axis is the vertically upward direction.

### Embodiment 1

### [Configuration]

First, a configuration of an awakening inducing system that includes an awakening inducing device according to Embodiment 1 will be described.

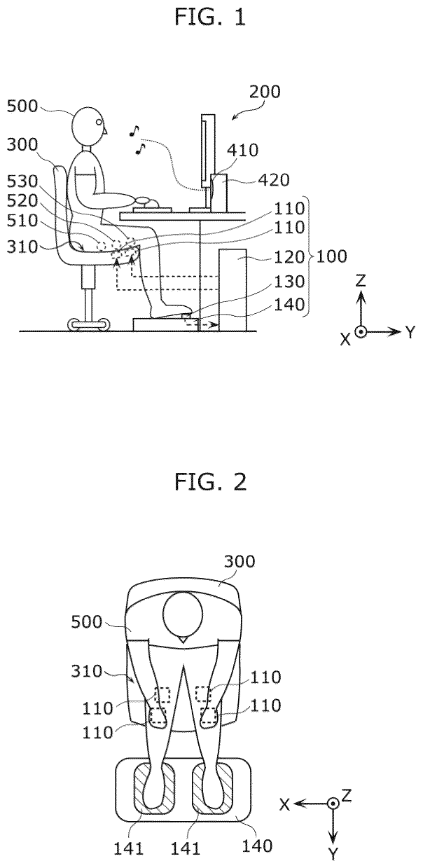

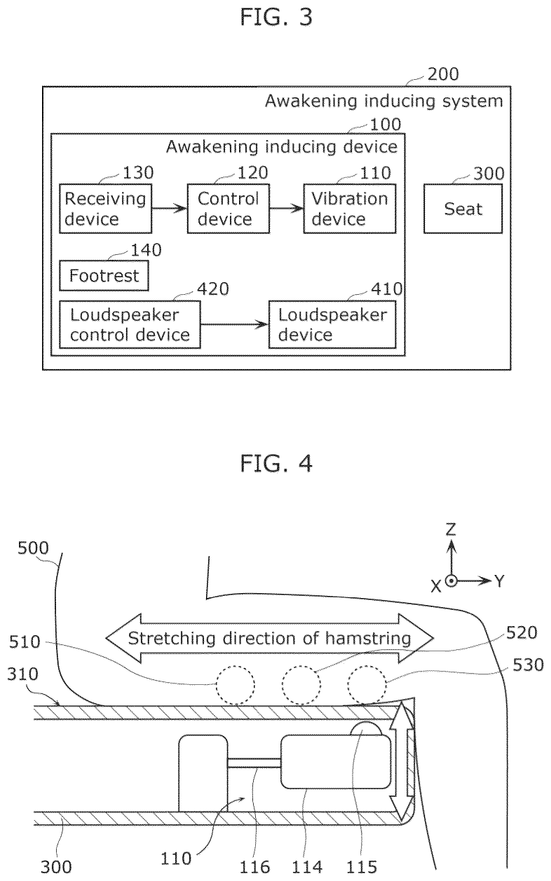

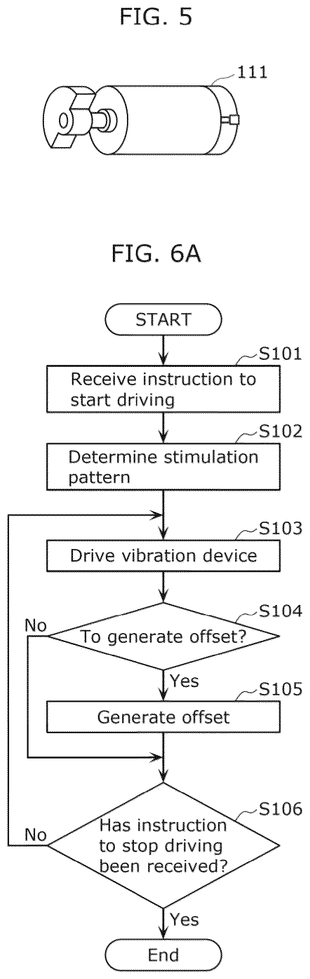

FIG. 1 is a side view illustrating awakening inducing system 200 according to Embodiment 1. FIG. 2 is a top view for explaining a positional relationship between vibration devices 110 according to Embodiment 1 and user 500. FIG. 3 is a block diagram illustrating a functional configuration of awakening inducing system 200 according to Embodiment 1. FIG. 4 is an illustration for explaining a position on user 500 to which vibration devices 110 according to Embodiment 1 give vibration stimulation. FIG. 4 illustrates only a portion of the inside of seat 300 for description. FIG. 5 is a diagram illustrating an example of eccentric motor 111 included in each vibration device 110. FIG. 1 and FIG. 2 each show a plurality of vibration devices 110, whereas FIG. 3 and FIG. 4 each show only one vibration device 110 for description. There is no particular limitation on the number of vibration devices 110 to be included in awakening inducing device 100.

Awakening inducing system 200 is a system that awakens user 500 by giving vibration stimulation to at least one of muscle belly portion 520 or insertion portion 530 of one or each of the hamstrings of user 500 sitting on seat 300 disposed in a mobile body, such as an automobile, or in an office.

Awakening inducing system 200 induces awakening of user 500 by giving vibration stimulation to user 500 who is experiencing a decrease in efficiency or facing a possible safety concern due to drowsiness while, for example, working at a desk, studying, or driving. It should be noted that the following describes that the vibration stimulation is given to at least one of muscle belly portion 520 or insertion portion 530 of hamstring, but the portion to which the vibration stimulation is given is not limited to the above portion. The portion to which the vibration stimulation is given may be any portion on user 500, such as a waist portion, a shoulder portion, and a foot portion (in particular, a sole of the foot, or the like) of user 500.

Awakening inducing system 200 includes awakening inducing device 100 and seat 300.

Awakening inducing device 100 is a device that awakens user 500 by giving vibration stimulation to at least one of muscle belly portion 520 or insertion portion 530 of one or each of the hamstrings of user 500 sitting on seat 300 disposed in a mobile body, such as an automobile, or in an office. In this example, awakening user 500 means at least one of bringing the state of user 500 to the one in which user 500 is more awake than in the current state or maintaining the awake state of user 500. In other words, awakening user 500 can mean eliminating drowsiness of user 500 (i.e., lowering the degree of drowsiness (the level of drowsiness)) or keeping user 500 from becoming drowsy (i.e., keeping the degree of drowsiness from rising). In the following description, either case is simply expressed as awakening user 500.

There is no particular limitation on the shape of seat 300 as long as seat 300 includes seating surface 310 on which user 500 sits. Seating surface 310 may be made of a material that can be deformed (elastically deformed) by vibration devices 110. Seating surface 310 is formed, for example, of a cushiony material.

Awakening inducing device 100 includes vibration devices 110, control device 120, receiving device 130, footrest 140, loudspeaker device 410, and loudspeaker control device 420. Control device 120 is an example of the controller, and configured to achieve the function of a part of the controller. Control device 120 is connected to each vibration device 110 and receiving device 130 such that these connected devices can transmit or receive signals therebetween via a wired circuit or a wireless circuit.

Vibration devices 110 is an example of the second awakener that induces awakening of user 500 by giving the stimulus of the second type to user 500. Vibration devices 110 are each a device that vibrates to give vibration stimulation to at least one of muscle belly portion 520 or insertion portion 530. Each vibration device 110 is disposed in seat 300 at a position where vibration device 110 opposes at least one of muscle belly portion 520 or insertion portion 530 of a hamstring of user 500 sitting on seat 300. According to the present embodiment, vibration devices 110 are disposed inside seat 300 and disposed at respective positions where vibration devices 110 can give vibration stimulation to user 500 via seating surface 310. In other words, vibration devices 110 are provided in seat 300 at respective positions where vibration devices 110 oppose user 500 via seating surface 310.

A hamstring is a collective term for a lower limb posterior thigh muscle and includes a biceps femoris, a semimembranosus, and a semitendinosus. A hamstring is divided into origin portion 510, muscle belly portion 520, and insertion portion 530, which are located at mutually different positions. For example, when a hamstring is equally divided into three parts along the stretching direction (the extending direction) of the hamstring, origin portion 510, muscle belly portion 520, and insertion portion 530 are located in this order from the side of the gluteus maximus toward the knee. Vibration devices 110 give vibration stimulation to at least one of muscle belly portion 520 or insertion portion 530, that is, give vibration stimulation to a hamstring at a position in the two-thirds of the hamstring closer to the side of the knee.

Vibration devices 110 give vibration stimulation to at least one of muscle belly portion 520 or insertion portion 530 of one or each of the hamstrings of user 500 sitting on seat 300 by, for example, vibrating in a vibration direction (the direction parallel to the Z-axis direction according to the present embodiment) that is parallel to the direction orthogonal to the stretching direction of the hamstrings (as illustrated in FIG. 4, the stretching direction is the direction parallel to the Y-axis, according to the present embodiment). The vibration direction of vibration devices 110 is, for example, the direction orthogonal to seating surface 310. For example, in a case where seating surface 310 of seat 300 lies in a plane parallel to the horizontal direction, vibration devices 110 each vibrate in the vertical direction. In other words, in this case, the vibration direction of each vibration device 110 is the vertical direction. To rephrase, vibration devices 110 are disposed in seat 300 such that their vibration direction extends in the vertical direction. A vertically downward force is constantly applied to the body of user 500 due to the gravitational force. When vibration stimulation in the vertical direction is given to user 500, the vibration direction coincides with the direction of the force that acts on the body of user 500. This configuration makes it possible to give vibration stimulation efficiently to at least one of muscle belly portion 520 or insertion portion 530 of user 500 while the body of user 500 remains in contact with seat 300 with an appropriate pressure.

In order to activate the supplementary motor area, the muscle spindles of the hamstrings need to be activated. The muscle spindles become activated as the muscle fibers of the hamstrings are stretched. Therefore, when vibration devices 110 vibrate in the direction perpendicular to the stretching direction of the hamstrings (specifically, the extending direction of the muscle fibers of the hamstrings) to give vibration stimulation to the hamstrings, the muscle spindles can be activated effectively. For example, if the muscle fibers are seen as a string, the string can be stretched more effectively when the string is pulled in the direction orthogonal to the extending direction of the string than when the string is pulled in the extending direction of the string. Hence, the string can be made to vibrate effectively. In other words, when vibration stimulation is given to the muscle fibers in the direction orthogonal to the extending direction of the muscle fibers, the vibration stimulation can be transmitted to the muscle spindles effectively and noninvasively (i.e., the vibration stimulation can be given not directly to the muscle spindles but to the muscle spindles via the skin surface).

It is to be noted that there is no particular limitation on the cycle of vibrations of each vibration device 110. This cycle of vibrations is, for example, from 40 Hz to 120 Hz. The cycle of vibrations may be from 60 Hz to 70 Hz. With such a cycle of vibrations, vibration devices 110 can awaken user 500 more effectively (e.g., more clearly). Furthermore, a more appropriate example of the cycle of vibrations by vibration device 110 will be described below together with the processing performed by awakening inducing system 200.

Vibration devices 110 each include, for example, eccentric motor 111 for generating vibrations, housing 114, projection portion 115, and leaf spring 116.

Eccentric motor 111 is a motor for generating vibrations. For example, eccentric motor 111 is electrically connected to a power source (not illustrated), such as a battery, via a power source cord or the like. Eccentric motor 111 is driven as the power from the power source is supplied to eccentric motor 111. Specifically, eccentric motor 111 is fixed to housing 114 inside housing 114. Eccentric motor 111 causes housing 114 to vibrate and thus gives vibration stimulation to user 500. Eccentric motor 111 can cause housing 114 to vibrate in one direction. Therefore, eccentric motor 111 can give vibration stimulation to user 500 in the vertical direction.

In this example, vibration devices 110 may each include, in place of eccentric motor 111, a rotary motor with a cam mechanism, a voice coil motor, a linear drive motor, or a rotary motor with a crank mechanism. Any of these motors can change the vibration direction of each vibration device 110 as desired in accordance with how these motors are installed.

Housing 114 is a box that houses eccentric motor 111. There is no particular limitation on the material, the shape, and so on of housing 114. Housing 114 includes, for example, projection portion 115 projecting in the outward direction from housing 114.

Projection portion 115 is a portion of housing 114 that projects in the outward direction from housing 114. The presence of projection portion 115 on housing 114 enables vibration devices 110 to give vibration stimulation appropriately to a specific area (portion) on user 500 (at least one of muscle belly portion 520 or insertion portion 530 of user 500). Each housing 114 is fixed to seat 300 via leaf spring 116, for example.

In this example, there is no particular limitation on the size of each projection portion 115. The size of each projection portion 115 is, for example, from 78 mm² (corresponding to φ10 mm) to 7,854 mm² (corresponding to φ100 mm) in terms of the area of projection portion 115 along a plane perpendicular to the vibration direction of vibration devices 110.

Moreover, there is no particular limitation on the shape of each projection portion 115. Each projection portion 115 may have a shape with a corner or a round shape, for example. Projection portion 115 may have a shape of a cylindroid, a shape of a circular column, a shape of a chamfered cylindroid or a chamfered circular column, or a shape of a dome. When projection portion 115 has any of such shapes, as compared with a case where projection portion 115 has a corner, for example, user 500 can be kept from feeling discomfort as projection portion 115 is pressed against user 500 with the stress concentrated at a very small area on user 500 where user 500 is pressed by projection portion 115.

Leaf spring 116 is a flat spring connecting housing 114 to seat 300. For example, one end of leaf spring 116 is connected to housing 114, and the other end that is the end opposite to the one end is connected to seat 300. Leaf spring 116 is disposed such that the direction normal to leaf spring 116 extends parallel to the vibration direction of housing 114, for example. With this configuration, housing 114 connected to leaf spring 116 vibrates in the direction parallel to the direction normal to leaf spring 116.

Control device 120 is a device that, by controlling vibration devices 110, causes vibration devices 110 to give vibration stimulation to at least one of muscle belly portion 520 or insertion portion 530.

Control device 120 includes, for example, a communication interface for transmitting or receiving a signal to or from each vibration device 110 or receiving device 130, a memory such as a flash memory or a hard disk drive (HDD) storing a control program, and a central processing unit (CPU) that executes the control program.

Receiving device 130 is a device that receives an instruction from user 500. Specifically, receiving device 130 receives, from user 500, an instruction that causes control device 120 to perform at least one of starting or stopping control of causing vibration devices 110 to give vibration stimulation. There is no particular limitation on receiving device 130 as long as receiving device 130 can detect a predetermined operation of user 500 indicating an instruction that causes control device 120 to perform at least one of starting or stopping control of driving vibration devices 110. Receiving device 130 is, for example but not limited to, a mechanical switch such as a push button that user 500 can depress or a sensor that detects a foot of user 500 placed on footrest 140. Receiving device 130 may be a mechanical locker switch that is disposed on footrest 140 and that user 500 can switch on or off based on the angle at which user 500 steps on the locker switch (i.e., user 500 can provide an instruction indicating whether vibration devices 110 should start or stop vibrating). Alternatively, receiving device 130 may be a mechanical button switch that can be switched on or off based on how far the switch is depressed. Based on the instruction that receiving device 130 has received, control device 120 performs at least one of starting or stopping control of causing vibration devices 110 to give vibration stimulation.

According to the present embodiment, receiving device 130 is disposed in or on footrest 140.

Footrest 140 is a support on which user 500 places at least one foot. Footrest 140 is disposed at a position where, when user 500 sitting on seat 300 has placed a foot or feet on footrest 140, at least one of muscle belly portion 520 or insertion portion 530 is in contact with seat 300 (seating surface 310 of seat 300, to be more specific). For example, marker portions 141 are provided on footrest 140 so as to indicate the positions where user 500 should place his or her feet. As long as user 500 can recognize marker portions 141, marker portions 141 may be marked on footrest 140 by being colored in a color different from the color of the rest of footrest 140 or marked in footrest 140 by a distinguishing shape, such as a concave shape or a convex shape, formed in footrest 140.

Loudspeaker device 410 is a device that applies a stimulus of a first type. Loudspeaker device 410 is an example of a first awakener that induces awakening of user 500. Loudspeaker device 410 is a device that emits sound. By controlling loudspeaker device 410 with consecutive input signals in accordance with a musical piece, loudspeaker device 410 can play back the musical piece. Specifically, loudspeaker device 410 includes, for example, a vibration plate and a drive mechanism including a magnet and a voice coil. Loudspeaker device 410 operates the drive mechanism with a waveform signal of the musical piece that is decomposed in the time domain, and the drive mechanism vibrates the vibration plate. In this manner, loudspeaker device 410 generates an acoustic wave with the vibration of the vibration plate in accordance with the musical piece, the acoustic wave propagates through the air to reach the ears of user 500, and thus user 500 perceives the musical piece.

Loudspeaker control device 420 is an example of a controller and has a configuration that implements some of functions of the controller. Loudspeaker control device 420 is a device that controls loudspeaker device 410 to cause loudspeaker device 410 to play back the musical piece.

For example, loudspeaker control device 420 obtains the consecutive input signals in accordance with the musical piece and outputs the input signals to loudspeaker device 410. By preprocessing the input signals to loudspeaker device 410 that are to be output from loudspeaker control device 420, loudspeaker control device 420 can play back the musical piece in any form.

### [Processing Procedure]

Next, processing procedures of awakening inducing system 200 according to Embodiment 1 will be described.

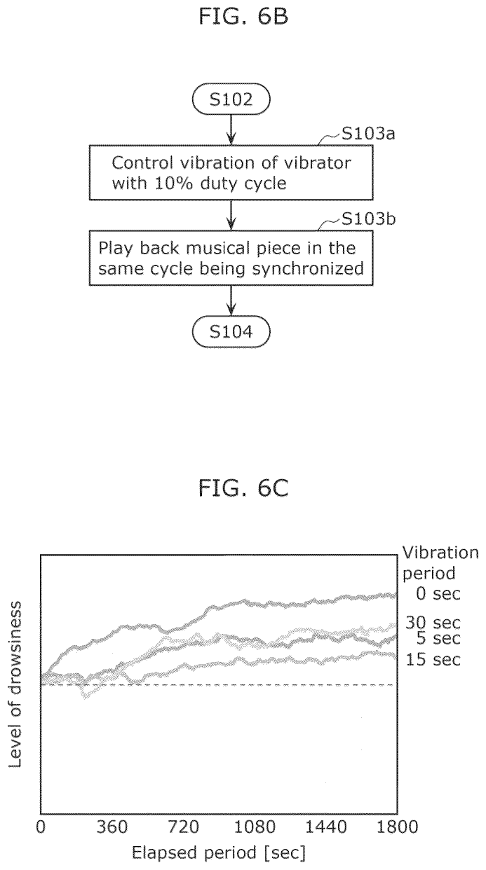

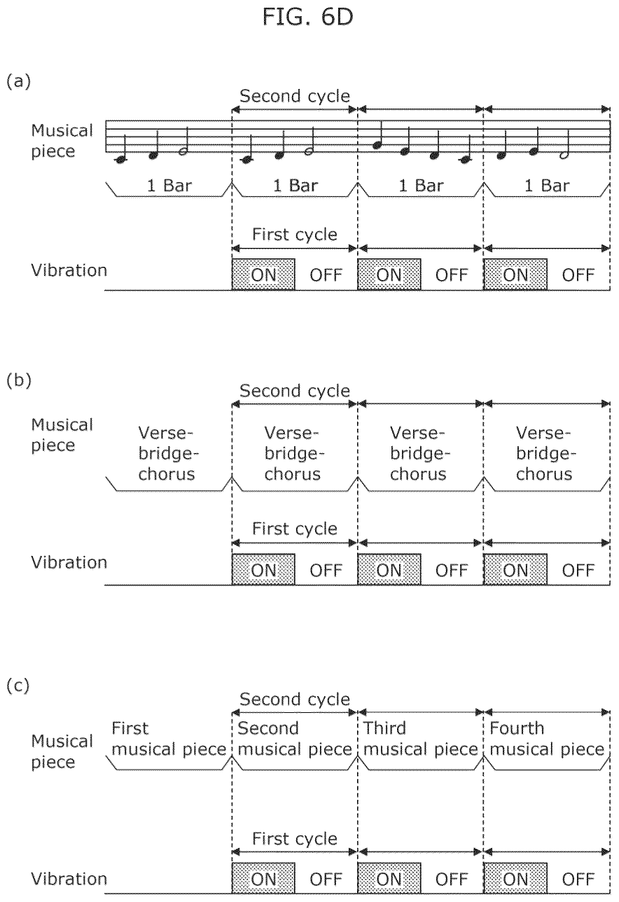

FIG. 6A is a flowchart for explaining a process executed by awakening inducing system 200 (specifically, awakening inducing devices 100) according to Embodiment 1.

First, receiving device 130 receives an instruction from user 500 that causes vibration devices 110 to start being driven (i.e., causes vibration devices 110 to start vibrating) (step S101). For example, in a case where receiving device 130 is a push button, user 500 has depressed the push button.

Next, control device 120 determines a stimulation pattern of vibration stimulation to be given to user 500 (step S102). Determining a stimulation pattern includes, for example, determining the cycle of vibrations, the amplitude of vibrations, or the position on user 500 to be stimulated. For example, if user 500 is on a task, such vibration stimulation that does not startle user 500 may be given to user 500. Specific examples of a vibration pattern that is less likely to startle user 500 includes (i) a vibration pattern in which the amplitude of vibrations of each vibration device 110 is increased gradually, (ii) a vibration pattern in which the cycle of vibrations of each vibration device 110 is increased gradually, or (iii) a vibration pattern in which the intervals of vibrations of each vibration device 110 are reduced gradually (i.e., the driving frequency is increased gradually). Awakening inducing device 100 includes, for example, an operator (not illustrated), such as a keyboard or a touch panel, to be operated by user 500. Control device 120, for example, obtains information indicating a vibration pattern obtained from user 500 via the operator and determines a vibration pattern based on the obtained information.

Next, control device 120 performs control of driving vibration devices 110 such that vibration devices 110 each vibrate in the vibration pattern determined at step S102 (step S103). With this control, control device 120, by controlling vibration devices 110, causes vibration devices 110 to give vibration stimulation to at least one of muscle belly portion 520 or insertion portion 530 via seating surface 310 of seat 300.

Here, FIG. 6B is a flowchart for explaining a part of the process executed by the awakening inducing system according to Embodiment 1. As illustrated in FIG. 6B, in the present embodiment, user 500 is awakened by sound and vibration by controlling loudspeaker device 410 in combination with driving vibration devices 110. Specifically, step S103 in FIG. 6A described above includes step S103a and step S103b illustrated in FIG. 6B.

As illustrated in FIG. 6B, when control device 120 performs control to drive vibration devices 110, control device 120 first controls the vibration of vibration devices 110 such that a duty cycle that represents a ratio of a vibration period during which vibration devices 110 vibrate to the sum of the vibration period and an interval time during which the vibration of vibration devices 110 is stopped becomes 10% (step S103a). As described above, in a set that includes one vibration period during which vibration devices 110 vibrate and one interval time that follows the vibration period and during which the vibration of vibration devices 110 is stopped, the duty cycle is the ratio of the vibration period to a period during which this set is executed. For example, the duty cycle is expressed as a percentage. The duty cycle being 10% is, for example, a case of the interval time being 270 seconds with respect to the vibration period being 30 seconds, a case of the interval time being 135 seconds with respect to the vibration period being 15 seconds, or a case of the interval time of 45 seconds with respect to the vibration period of 5 seconds. By controlling vibration devices 110 in this manner such that the duty cycle becomes 10%, the effect (efficiency) of inducing the awakening of user 500 is improved. When the correlation between the duty cycle and the awakening effect on user 500 is assumed to be a normal-distribution-like relationship whose peak is at 10%, it is considered that even the duty cycle not being 10% can produce a relatively high awakening effect. That is, control device 120 may control the vibration of vibration devices 110 such that the duty cycle becomes 15% or may control the vibration of vibration devices 110 such that the duty cycle becomes 5%. Within such a range of the duty cycle, it is expected that a high awakening effect is produced.

With the duty cycle being 10%, the vibration period can be set variously as described above. This point will be described with reference to FIG. 6C. FIG. 6C is a graph for explaining an appropriate vibration period of the awakening inducing system according to Embodiment 1. The vertical axis represents the level of drowsiness, on which an upward direction in the graph indicates a stronger drowsiness of a user, and a downward direction in the graph indicates a weaker drowsiness of the user. FIG. 6C illustrates changes in the level of drowsiness with respect to elapsed period when vibration stimuli are applied on user 500 under vibration conditions that the vibration period is set to 0 sec (no vibration), 5 sec, 15 sec, and 30 sec, with the duty cycle fixed to 10%. As illustrated in FIG. 6C, in a case of setting the vibration period to 15 sec and the interval time to 135 sec from among the vibration conditions, the level of drowsiness changes at lowest values, and it is found that this condition provides a high awakening effect. In addition, it is found that the awakening effect gradually increases when the vibration period is changed from 0 sec to 5 sec and from 5 sec to 15 sec, and it is found that the awakening effect gradually decreases when the vibration period is changed from 15 sec to 30 sec. Therefore, the correlation between the vibration period and the level of drowsiness is estimated as a normal-distribution-like relationship whose peak is in the vicinity of 15 sec. Thus, it is considered that the vibration period around 15 sec, for example, within the range between 10 seconds to 20 seconds inclusive produces a high awakening effect. In addition to this, it is considered that the interval time within the range between 130 seconds to 140 seconds inclusive produces a high awakening effect.

Referring back to FIG. 6B, while control device 120 controls the vibration of vibration devices 110, loudspeaker control device 420 causes loudspeaker device 410 to play back the musical piece (step S103b). In this situation, when a repetition cycle of a given section in the musical piece being played back is denoted as a second cycle, the second cycle and a first cycle, which is a cycle of repeating one vibration set including one vibration period and one interval time, are the same cycle being synchronized. To do this, at least one of control device 120 or loudspeaker control device 420 performs the control such that a repetition cycle of a control target becomes the same cycle being synchronized.

Here, the vibration is controlled along with the musical piece being played back in such a manner as to set the first cycle such that control device 120 controls vibration devices 110 in the same cycle being synchronized with the second cycle. Here, FIG. 6D is a diagram for explaining the relationship between the first cycle and the second cycle in the awakening inducing system according to Embodiment 1. In FIG. 6D, (a) to (c) each illustrate an example of the given section in the musical piece. For example, in (a) of FIG. 6D, the given section in the musical piece is set to be one bar, and a section in which this bar is repeated (alternatively, a section from the start of a bar to the start of the next bar) is determined as the second cycle. Control device 120 then controls the vibration of vibration devices 110 such that the one vibration set including one vibration period and one interval time is performed in the first cycle that is made to match the second cycle, that is, a period in which one bar in the musical piece is played back.

Likewise, for example, in (b) of FIG. 6D, the given section in the musical piece is set to be a section including a series of constitutional elements that include one verse block and one chorus block, and a section in which this section including the constitutional elements is repeated (alternatively, a section from the start of a section including the constitutional elements to the start of the next section including the constitutional elements) is determined as the second cycle. Control device 120 then controls the vibration of vibration devices 110 such that the one vibration set including one vibration period and one interval time is performed in the first cycle that is made to match the second cycle, that is, a period in which the section including the constitutional elements in the musical piece is played back.

Likewise, for example, in (c) of FIG. 6D, the given section in the musical piece is set to be a section from the start to the end of the entire musical piece, and a section in which this musical piece is repeated (alternatively, a section from the start of a musical piece to the start of the next musical piece) is determined as the second cycle. Control device 120 then controls the vibration of vibration devices 110 such that the one vibration set including one vibration period and one interval time is performed in the first cycle that is made to match the second cycle, that is, a period in which the musical piece is played back.

By alternating the vibration period and the interval time along with the given section in the musical piece in the above-described manner, user 500 can predict that the vibration stimulus is applied when a given section in the musical piece is switched to the following, next given section. Thus, it is possible to reduce the possibility that user 500 is surprised by a sudden vibration stimulus, which adversely affects work by user 500.

When the first cycle being the repetition cycle including the vibration period and the interval time is made to match the second cycle obtained from the given section of the musical piece, fixing the duty cycle to 10% or fixing the vibration period to 15 sec requires the adjustment of the given section in the musical piece. Thus, in order to fix the duty cycle to 10% or fix the vibration period to 15 sec, for example, loudspeaker control device 420 performs fade-out in which the playback of a musical piece is finished in the middle of a given section including two or more bars or a certain bar, and a sound volume is gradually reduced immediately before the playback is finished. Accordingly, it is possible to apply the vibration stimulus along with a given section in a musical piece while fixing the duty cycle to 10% or fixing the vibration period to 15 sec.

Here, when the playback of the musical piece and the vibration stimulus, that is, the two stimuli are applied to user 500 in the same cycle being synchronized as described above, it is possible to reduce a surprise to user 500 caused by the vibration stimulus. On the other hand, there is the risk that user 500 becomes desensitized to such application of the periodic stimulus, weakening the effect of awakening user 500. Hence, the following will describe a configuration capable of maintaining the high effect of awakening user 500 while reducing a surprise to user 500.

In the following description of the configuration, a combination of a stimulus whose type is sound, particularly the playback of a musical piece, and a stimulus whose type is vibration will be mentioned. However, a combination of types of stimuli is not limited to this. For example, the combination of types of stimuli may be a playback of a sound effect or the like, not a musical piece, out of stimuli whose type is sound, and a stimulus whose type is vibration. For example, the combination of types of stimuli may be a combination of a stimulus whose type is light, particularly a playback of a video, and a stimulus whose type is vibration. For example, the combination of types of stimuli may be a combination of a stimulus whose type is light (a playback of a video) and a stimulus whose type is vibration (a playback of a musical piece or a sound effect). For example, the combination of types of stimuli may be a combination of a stimulus whose type is vibration and a stimulus whose type is vibration. In a case of the same type of stimuli, the stimuli are different from each other in at least parts of their content. Examples of the stimuli include a combination of stimuli that are presented to portions different from each other, such as a combination of a stimulus whose type is vibration that vibrates a right leg and a stimulus whose type is vibration that vibrates a left leg.

For example, in the above example illustrated in FIG. 6D explains that the playback of the musical piece and the vibration stimulus are applied to user 500 in the same cycle being synchronized. The playback of the musical piece and the vibration stimulus mean that the periodic stimulus of which the start timings are synchronized with each other is repeated a plurality of times. In other words, a stimulus period during which the stimulus is applied (corresponding to the vibration period in the stimulus of the vibration described above) and a stop period during which no stimulus is applied (corresponding to the interval time in the stimulus of the vibration described above) are periodically alternated, and it can be considered that the start timings of the two stimuli in the stimulus period synchronized with each other each time.

The stop period in the playback of the musical piece refers to a period that is present immediately before the above-described switch from a given section to the next given section in the musical piece. This period is usually a silent period. It should be noted that, while the given section is set to be one bar, some musical piece or the like includes musical notes that are connected by a slur or a tie over two bars. In such a case, it is assumed that the stimulus period includes only an instant when the musical notes start and an instant when the musical notes stop. That is, in a case where musical notes connected by a slur or a tie continue over two or more given sections, only a timing at which a musical note changes from an immediately preceding musical note is assumed to be included in the stimulus period, and a period in which a musical note remains unchanged from an immediately preceding musical note is assumed to be included in the stop period in which no stimulus is applied even when the musical note is present in the period. Therefore, in the case of a stimulus whose type is sound, in the form of a playback of a musical piece, the stop period need not be a simply silent period.

Likewise, in the case of a playback of a video, in which the type of its stimuli is light, the stop period is a period that is present immediately before a given section is switched to the next given section in the video. The stop period may be a period with no signal or may be a period with a signal. Specifically, in the case of a stimulus given by playing back a video, a period with a signal may be simply taken as the stimulus period in which the stimulus is applied, and a period with no signal may be simply taken as the stop period in which the stimulus is not applied. However, for example, a period in which an average of luminance or the like in the whole video is at least a threshold value may be taken as the stimulus period, and a period in which the average of luminance or the like is less than the threshold value may be taken as the stop period in which the stimulus is not applied even when there is a signal in the period. Therefore, in the case of a stimulus whose type is light, in the form of a playback of a video, the stop period need not be a simply no-signal period.

In a case where the start timings are synchronized with each other each time, user 500 is eventually desensitized to the synchronization. Thus, the start of one of the stimuli can be predicted from the periodic repetition of another one of the stimuli. As a result, the repetitive stimulus is applied at repeated timings that are periodic and predictable, and thus the effect of awakening user 500 is weakened.

To resolve this, for example, the stimulus period and the stop period of each stimulus may be controlled such that the start timings are not synchronized in some of the plurality of the repeated cycles each including the stimulus period and the stop period. That is, the start timings may be offset from each other in one cycle of the plurality of cycles. In other words, an offset cycle in which the start timings are offset from each other may occur after a synchronization cycle in which the start timings are synchronized with each other is repeated a plurality of times.

In such an offset cycle, the offset needs to be perceivable by user 500. Thus, for example, the start timings may be offset from each other by a perceivable length in the time domain. The length of offset which makes the offset to be perceivable differs among types of stimulus. Thus, the length of offset may be set in accordance with types of stimuli. For example, in the present embodiment, the combination of the playback of the musical piece, that is, a sound stimulus and a vibration stimulus are used in combination. NPL 1 describes that an offset of at least 40 ms is perceivable. For this reason, the length of offset may be at least 40 ms. In contrast, too long the offset may disable the repetition of the periodic stimulus. Specifically, when it is assumed that a second stimulus is delayed from a first stimulus by the offset, if a stimulus period of a second stimulus in some cycle overlaps the next stimulus period (if a stop period disappears in the cycle), the periodic repetition is disabled. Hence, the length of offset may be at most half of the stop period in the cycle.

The pattern in which the offset cycle occurs with respect to the synchronization cycle may be determined in advance or may be at random. Specifically, the periodic stimulus is repeatedly applied, for example, in such a manner as to repeat a cycle pattern that includes a combination of one offset cycle and one or more synchronization cycles in a predetermined order. The predetermined order may be any order.

Specifically, the periodic stimulus is repeatedly applied, for example, in such a manner as to repeat a cycle pattern that includes one offset cycle and one or more synchronization cycles in a random order of the offset cycle and the synchronization cycles. In this case, the place of the offset cycle is not constant in some cycle pattern and the following cycle pattern. Alternatively, whether each cycle may be the offset cycle or the synchronization cycle may be determined simply at random.

Referring back to FIG. 6A, when the cycles of the stimuli enter the offset cycle in which the offset is generated (Yes in S104), the cycle of one of the stimuli is offset with respect to the cycle of another one of the stimuli in the time domain (S105). More specifically, control device 120 controls vibration devices 110 in such a manner as to cause the stimulus period in the cycle of the one of the stimuli (e.g., the vibration stimulus) to start delayed from the stimulus period in the cycle of the other one of the stimuli (the playback of the musical piece).

On the other hand, when the cycles of the stimuli enter the synchronization cycle in which the stimuli are synchronized with each other (the offset is not generated) (No in S104), step S105 is skipped, and the synchronized stimuli are applied without the generation of the offset.

Thereafter, control device 120 determines whether receiving device 130 has received an instruction from user 500 that causes vibration devices 110 to stop being driven (step S106).

When control device 120 determines that receiving device 130 has received an instruction from user 500 that causes vibration devices 110 to stop being driven (Yes in step S106), control device 120 causes vibration devices 110 to stop being driven and terminates the process. For example, in a case where receiving device 130 is a push button, control device 120 causes vibration devices 110 to stop being driven and terminates the process in response to user 500 depressing the push button the second time.

On the other hand, when control device 120 determines that receiving device 130 has not received any instruction from user 500 that causes vibration devices 110 to stop being driven (No in step S106), control device 120 returns the process to step S103.

Control device 120 may stop vibration devices 110 and terminate the process after vibration devices 110 have been driven for a predetermined time determined in advance. In this case, control device 120 may include a timer, such as a real time clock (RTC), for measuring the time. Accordingly, it is possible to restrain the effect of awakening user 500 by awakening inducing device 100 from decreasing as user 500 becomes desensitized to the vibration stimulus.

A continuous vibration stimulus may cause user 500 to pay constant attention to the vibration stimulus, and this may keep user 500 from concentrating on a task, such as studying. Therefore, awakening inducing device 100 may apply the vibration stimulus intermittently to user 500. For example, awakening inducing device 100 may apply the vibration stimulus to user 500 with intervals determined in advance.

### Other Advantageous Effects, Etc.

As described above, awakening inducing device 100 according to Embodiment 1 includes vibration devices 110 and control device 120. Vibration devices 110 are disposed in seat 300 at respective positions where vibration devices 110 oppose at least one of muscle belly portion 520 or insertion portion 530 of one or each of the hamstrings of user 500 sitting on seat 300. Control device 120, by controlling vibration devices 110, causes vibration devices 110 to apply the vibration stimulus to at least one of muscle belly portion 520 or insertion portion 530.

This configuration makes it possible to apply the vibration stimulus to at least one of muscle belly portion 520 or insertion portion 530 of user 500.

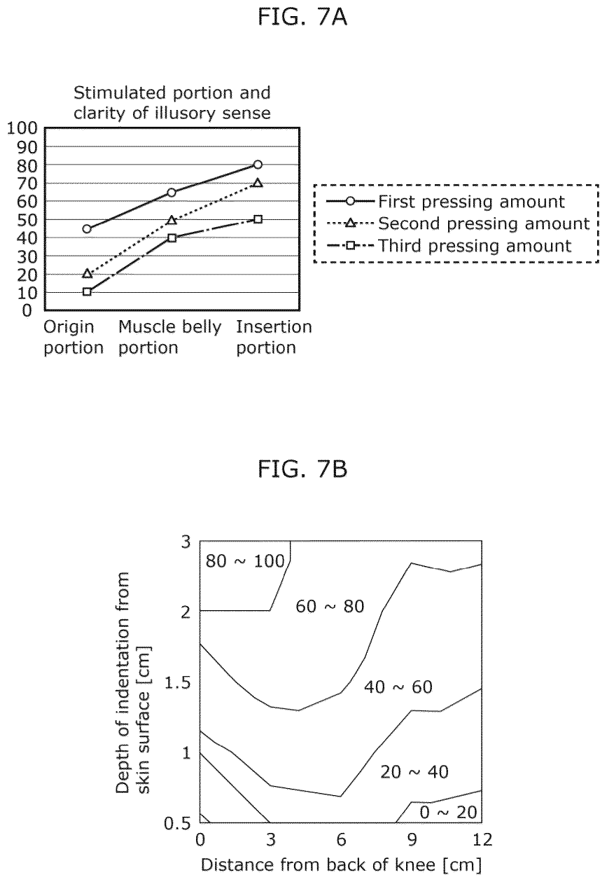

FIG. 7A is a graph for explaining effects of awakening inducing system 200 according to Embodiment 1. Specifically, FIG. 7A shows the clarity of illusory sense reported by subjects in an experiment in which the subjects were given vibration stimulation at each of the areas of their hamstrings. In FIG. 7A, the vertical axis represents the clarity of illusory sense, where 0 was assigned when no illusory sense was elicited and 100 was assigned when a very clear illusory sense was elicited. Values representing the clarity of illusory sense were obtained through self-reporting of the subjects, where the subjects reported the corresponding clarity along the continuous index axis. The vibration stimulation given to each area had a vibration frequency of 70 Hz and was given continuously for 10 seconds. The pressing amount is the amount that indicates how far each area to which vibration device 110 gave vibration stimulation was pressed. A first pressing amount was 1.8 cm, a second pressing amount was 1.3 cm, and a third pressing amount was 0.8 cm. Vibration devices 110 vibrated while being pressed into the body by any of the aforementioned pressing amounts.

As illustrated in FIG. 7A, a motion illusion is produced more clearly in muscle belly portion 520 or insertion portion 530 of the hamstrings than in origin portion 510. In other words, FIG. 7A shows that vibration stimulation has a high illusory effect when given to muscle belly portion 520 or insertion portion 530 of the hamstrings. Based on the above, the inventors of the present application have found that it is possible to awaken a user more fully than conventional techniques by giving vibration stimulation to at least one of muscle belly portion 520 or insertion portion 530 of user 500. In other words, awakening inducing device 100 can awaken user 500 more fully than conventional techniques by giving vibration stimulation to at least one of muscle belly portion 520 or insertion portion 530 of user 500.

For example, vibration device 110 is disposed in seat 300 at a position where vibration device 110 opposes insertion portion 530. In this case, control device 120 causes vibration device 110 to give vibration stimulation to insertion portion 530, for example.

This configuration makes it possible to give vibration stimulation to insertion portion 530 of the user. The inventors of the present application have found that it is possible to awaken user 500 more effectively by giving vibration stimulation particularly to insertion portion 530, out of muscle belly portion 520 and insertion portion 530, of user 500, as illustrated in FIG. 7A. In other words, such a configuration makes it possible to awaken user 500 more effectively by giving vibration stimulation to insertion portion 530 of user 500. For this example, results illustrated in FIG. 7B were obtained by an additional experiment. FIG. 7B is a graph for explaining, in more detail, the effects of the awakening inducing system according to Embodiment 1. FIG. 7B illustrates the results of a study of positions of a user to which the vibration stimulus is applied. In FIG. 7B, its horizontal axis represents the distance from a back of a knee to a position at which the vibration stimulus is applied when the position was moved to various positions in the stretching direction of the hamstring from the back of the knee. In FIG. 7B, its vertical axis represents the depth of indentation of the position at which the vibration stimulus is applied was indented in a direction intersecting a skin surface of user 500. This depth of indentation corresponds to each of the pressing amount in FIG. 7A. In FIG. 7B, relative values of the clarity of illusory sense are numerically shown in the graph. A clarity of illusory sense closer to 100 shows a higher awakening effect.

As illustrated in FIG. 7B, the shorter the distance in the stretching direction of the hamstring from the back of a knee to a position at which vibration stimulus is applied, the higher the clarity of illusory sense. For example, it is found that applying the vibration stimulus at a position at most 6 cm from the back of the knee in the stretching direction of the hamstring provides a clarity of illusory sense as high as 80 to 100. In addition, as illustrated in FIG. 7B, the larger the depth of indentation of the position at which the vibration stimulus is applied in the direction intersecting the skin surface, the higher the clarity of illusory sense. For example, it is found that applying the vibration stimulus at a position of a depth of indentation of at least 2 cm in the direction intersecting the skin surface provides a clarity of illusory sense as high as 80 to 100.

For example, awakening inducing device 100 further includes footrest 140 on which user 500 places at least one foot. In this case, footrest 140 is disposed at a position where, when user 500 sitting on seat 300 has placed a foot on footrest 140, at least one of muscle belly portion 520 or insertion portion 530 is in contact with seat 300.

With this configuration, when footrest 140 is disposed at an appropriate position and when user 500 has placed one or both of his or her feet on footrest 140, user 500 can be brought into such a posture that allows vibration devices 110 to apply the vibration stimulus appropriately to at least one of muscle belly portion 520 or insertion portion 530. Therefore, this configuration makes it possible to apply the vibration stimulus appropriately to at least one of muscle belly portion 520 or insertion portion 530 of user 500.

For example, awakening inducing device 100 further includes receiving device 130 that receives an instruction. In this case, receiving device 130 is disposed in or on footrest 140. For example, based on the instruction that receiving device 130 has received, control device 120 performs at least one of starting or stopping control of causing vibration devices 110 to apply the vibration stimulus.

Accordingly, the vibration stimulus is restrained from being applied user 500 at a timing that is not intended by user 500. For example, if the vibration stimulus is applied to user 500 at a timing that is not intended by user 500 while user 500 is driving, this vibration stimulus may surprise user 500, causing dangerous driving. As such, user 500 selects, via receiving device 130, the timing at which the vibration stimulus is applied to user 500 as desired, and this can keep user 500 from being surprised by the vibration stimulus applied at an unintended timing.

### Embodiment 2

Next, a configuration of an awakening inducing system that includes an awakening inducing device according to Embodiment 2 will be described. The description of the awakening inducing system according to Embodiment 2 centers on the differences from awakening inducing system 200 according to Embodiment 1. In the description of the awakening inducing system according to Embodiment 2, configurations identical to those of awakening inducing system 200 according to Embodiment 1 are given identical reference characters, and their description will be partly simplified or omitted.

### [Configuration]

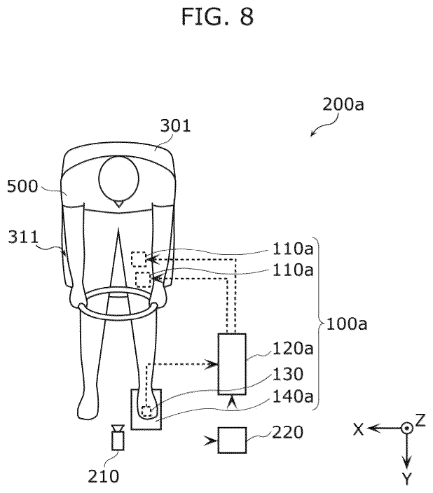

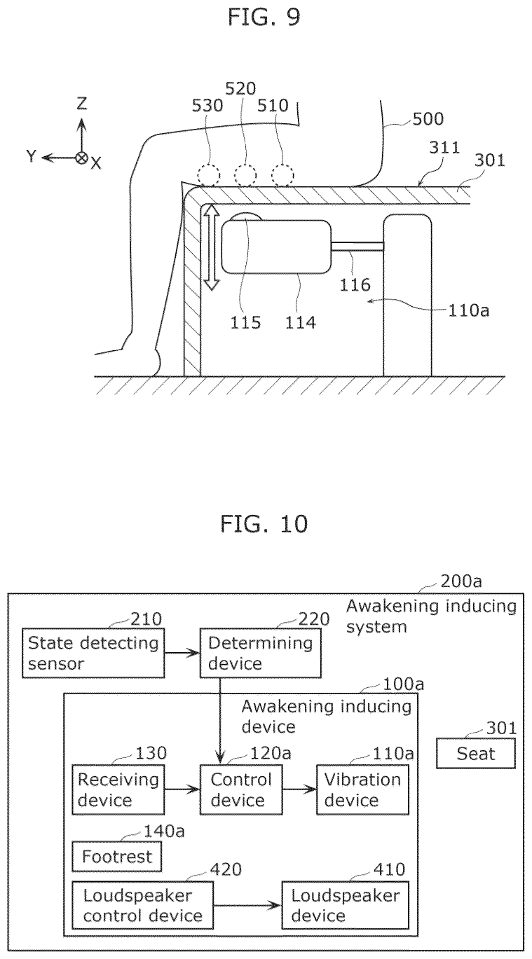

FIG. 8 is a top view illustrating awakening inducing system 200a according to Embodiment 2. FIG. 9 is a diagram for explaining a positional relationship between vibration device 110a according to Embodiment 2 and user 500. FIG. 10 is a block diagram illustrating a functional configuration of awakening inducing system 200a according to Embodiment 2. FIG. 8 shows two vibration devices 110a, whereas FIG. 10 shows only one vibration device 110a for description.

Awakening inducing system 200a includes awakening inducing device 100a, seat 301, state detecting sensor 210, and determining device 220.

Awakening inducing device 100a is a device that awakens user 500 by giving vibration stimulation to at least one of muscle belly portion 520 or insertion portion 530 of a hamstring of user 500 sitting in seat 301. According to the present embodiment, seat 301 is a driver's seat of a vehicle, such as an automobile. User 500 is the driver sitting in seat 301 serving as the driver's seat.

There is no particular limitation on the shape of seat 301 as long as seat 301 includes seating surface 311 on which user 500 sits. Seating surface 311 may be made of a material that can be deformed (elastically deformed) by vibration devices 110a. Seating surface 311 is formed, for example, of a cushiony material.

Awakening inducing device 100a includes vibration devices 110a, control device 120a, receiving device 130, and footrest 140a. Control device 120a is connected to each vibration device 110a and receiving device 130 such that these connected devices can transmit or receive signals therebetween via a wired circuit or a wireless circuit.

Vibration devices 110a are each a device that gives vibration stimulation to at least one of muscle belly portion 520 or insertion portion 530.

According to the present embodiment, vibration devices 110a are disposed at respective positions where vibration devices 110a oppose only the left leg of user 500 sitting in seat 301. Specifically, vibration device 110a is provided in seat 301 for the user at at least one of a position where vibration device 110a opposes muscle belly portion 520 of the hamstring of only the left leg of user 500 sitting in seat 301 or a position where vibration device 110a opposes insertion portion 530 of the hamstring of only the left leg of user 500 sitting in seat 301. In other words, each vibration device 110a is disposed in seat 301 such that vibration device 110a gives vibration stimulation to the left leg of user 500 but not to the right leg of user 500.

Vibration devices 110a give vibration stimulation to at least one of muscle belly portion 520 or insertion portion 530 of the hamstring of the left leg of user 500 sitting in seat 301 by, for example, vibrating in a vibration direction that is parallel to the direction orthogonal to the extending direction of the hamstring.

According to the present embodiment, vibration devices 110a are disposed in seat 301 between seating surface 311 and the floor surface. It suffices that vibration devices 110a each be provided in seat 301 at at least one of a position where vibration device 110a opposes muscle belly portion 520 of the hamstring of user 500 sitting in seat 301 or a position where vibration device 110a opposes insertion portion 530 of the hamstring of user 500 sitting in seat 301, and vibration devices 110a may be provided inside seat 301, under seat 301, or on top of or above seat 301. It suffices that vibration devices 110a be disposed at respective positions where vibration devices 110a can give vibration stimulation to user 500 via seating surface 311 of seat 301 in which user 500 sits.

Control device 120a is a device that, by controlling vibration devices 110a, causes vibration devices 110a to give vibration stimulation to at least one of muscle belly portion 520 or insertion portion 530 of the left leg of user 500.

Control device 120a includes, for example, a communication interface for transmitting or receiving a signal to or from each vibration device 110a or receiving device 130, a memory such as a flash memory or an HDD storing a control program, and a CPU that executes the control program.

Footrest 140a is a support on which user 500 places at least one foot. According to the present embodiment, footrest 140a is disposed at a position where user 500 places his or her left foot. Specifically, footrest 140a is disposed at a position where user 500 sitting in seat 301 places his or her left foot. In other words, footrest 140a is disposed at a position where user 500 places his or her left foot and not where user 500 places his or her right foot.

Footrest 140a is provided at a position where, when user 500 sitting in seat 301 has placed the left foot on footrest 140a, at least one of muscle belly portion 520 or insertion portion 530 is in contact with seating surface 311 of seat 301.

State detecting sensor 210 is a sensor for detecting state information that indicates a state of user 500 sitting in seat 301. Specifically, state detecting sensor 210 is a sensor for detecting the degree of drowsiness of user 500. State detecting sensor 210 is, for example, a camera.

Determining device 220 is a processing device that determines the degree of drowsiness (also referred to as “the level of drowsiness”) of user 500 based on the state information detected by state detecting sensor 210. Specifically, determining device 220 calculates the degree of drowsiness of user 500. A set of state detecting sensor 210 and determining device 220 is an example of the drowsiness estimator.

For example, state detecting sensor 210 detects a moving image including the face of user 500 as state information. For example, determining device 220 is connected to and can communicate with state detecting sensor 210, and determining device 220 determines the degree of drowsiness of user 500 based on a moving image including user 500 captured by state detecting sensor 210.

Determining device 220 determines the degree of drowsiness of user 500 in a five point scale. For example, determining device 220 determines that the degree of drowsiness is low when user 500 blinks at regular intervals (i.e., user 500 is not drowsy) and determines that the degree of drowsiness is one. Meanwhile, determining device 220 determines that the degree of drowsiness is high when user 500 blinks slowly and frequently at short intervals (i.e., user 500 is somewhat drowsy) and determines that the degree of drowsiness is, for example, three. In other words, user 500 is determined not to be drowsy when user 500 blinks at regular intervals and determined to be drowsy when user 500 blinks slowly and frequently. In this manner, determining device 220 detects the degree of drowsiness of user 500 by analyzing a moving image including user 500 obtained from state detecting sensor 210. The degree of drowsiness determined by determining device 220 may be a degree of drowsiness at a time point when state detecting sensor 210 detects the state information, a change degree (in other words, a degree of a temporal change) of drowsiness between time points including the time point when state detecting sensor 210 detects the state information, or a (future) degree of drowsiness after the time point when state detecting sensor 210 detects the state information.

In this example, the degree of drowsiness may be classified in a six or more point scale or a four or less point scale.

In one example, the user may be determined to be less drowsy as the degree of drowsiness of user 500 that determining device 220 calculates in the form of a numerical value is higher.

In the following description, the assumption is that the drowsiness is stronger as the degree of drowsiness of user 500 that determining device 220 calculates in the form of a numerical value is higher.

Determining device 220 includes, for example, a communication interface for obtaining (receiving) information that indicates the state of user 500 from state detecting sensor 210, a memory such as a flash memory or an HDD storing a control program, and a CPU that executes the control program.

In this example, control device 120a and determining device 220 may each be implemented by a separate dedicated circuit or a separate CPU or by a single dedicated circuit or a single CPU.

There is no particular limitation on how the degree of drowsiness of user 500 is determined. For example, state detecting sensor 210 may be a sensor that detects the pressure distribution across seating surface 311 on which user 500 sits. In this case, determining device 220, for example, calculates the amount of change in the centroid position of user 500 based on the pressure distribution across seating surface 311 on which user 500 sits and determines the degree of drowsiness of user 500 based on the calculated amount of change.

In this manner, state detecting sensor 210 does not need to be a camera and may be, for example but not limited to, a pressure distribution sensor, a motion sensor, a heartbeat sensor, a pulse sensor, or a breathing sensor.

The degree of drowsiness that determining device 220 determines may be the degree of drowsiness held at a desired point in time between the past and the current time calculated based on the state information accumulated in the past and the current state information or may be the degree of drowsiness to be held at a desired point in time in the future calculated based on the state information accumulated in the past and the current state information.

### [Processing Procedure]

Next, processing procedures of awakening inducing system 200a according to Embodiment 2 will be described.

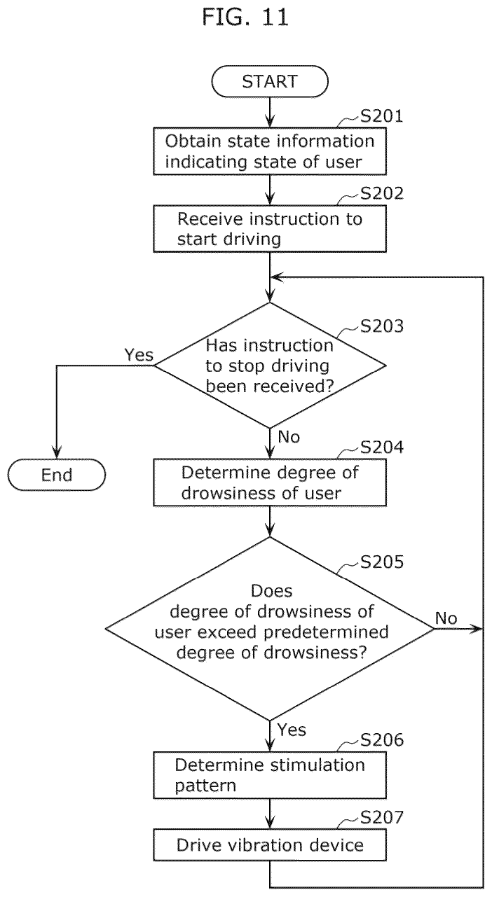

FIG. 11 is a flowchart for explaining a process executed by awakening inducing system 200a according to Embodiment 2.

First, determining device 220 obtains information indicating the state of user 500 from state detecting sensor 210 (step S201). In a case where state detecting sensor 210 is a camera, determining device 220 obtains, as information indicating the state of user 500, a moving image of user 500 from state detecting sensor 210, for example.

Next, receiving device 130 receives an instruction from user 500 (step S202). For example, in a case where receiving device 130 is a push button, user 500 has depressed the push button.

In this example, state detecting sensor 210 may keep transmitting information indicating the state of user 500 to determining device 220 at desired timings. Alternatively, state detecting sensor 210 may transmit information indicating the state of user 500 to determining device 220 in response to, for example, receiving a signal that requests information indicating the state of user 500 from determining device 220. Determining device 220 may transmit a signal that requests information indicating the state of user 500 to state detecting sensor 210 in response to receiving device 130 receiving an instruction from user 500. Determining device 220 may receive information indicating that receiving device 130 has received an instruction from user 500 from control device 120a or from receiving device 130. In other words, step S201 may be executed after step S202.

Next, control device 120a determines whether receiving device 130 has received an instruction from user 500 that causes vibration devices 110a to stop being driven (step S203).

If control device 120a determines that receiving device 130 has received an instruction from user 500 that causes vibration devices 110a to stop being driven (Yes at step S203), control device 120a causes vibration devices 110a to stop being driven and terminates the process if vibration devices 110a are being driven or simply terminates the process if vibration devices 110a are not being driven.

Meanwhile, if control device 120a determines that receiving device 130 has not received any instruction from user 500 that causes vibration devices 110a to stop being driven (No at step S203), determining device 220 determines the degree of drowsiness of user 500 based on the state information indicating the state of user 500 obtained from state detecting sensor 210 (step S204).

Next, determining device 220 determines whether the degree of drowsiness of user 500 determined at step S204 exceeds a predetermined degree of drowsiness (step S205). There is no limitation on the predetermined degree of drowsiness, and the predetermined degree of drowsiness may be set in advance as desired.

If determining device 220 determines that the degree of drowsiness of user 500 does not exceed the predetermined degree of drowsiness, in other words, the state of drowsiness of user 500 is closer to an awake state than a threshold state is (No at step S205), control device 120a returns the process to step S203.

Meanwhile, if determining device 220 determines that the degree of drowsiness of user 500 exceeds the predetermined degree of drowsiness, in other words, the state of drowsiness of user 500 is closer to an asleep state than the threshold state is (Yes at step S205), control device 120a determines a stimulation pattern (stimulation condition) of vibration stimulation to be given to user 500 (step S206). Determining a stimulation pattern includes, for example, determining the period of vibrations (in other words, the length of vibrations), the frequency of vibrations, the amplitude of vibrations (in other words, the intensity of vibrations), or the position on user 500 to be stimulated. Furthermore, the determining a stimulation pattern may include determining as to whether or not to include an offset cycle in the repeated cycles. If an offset cycle is not to be included in the repeated cycles, loudspeaker device 410 and vibration device 110 are controlled so that the repeated cycles include synchronization cycles only. This may be performed when the state of drowsiness of user 500 is closer to the awake state than the threshold state is. On the other hand, if an offset cycle is to be included in the repeated cycles, loudspeaker device 410 and vibration device 110 are controlled so that the repeated cycles include both an offset cycle and a synchronization cycle. This may be performed when the state of drowsiness of user 500 is closer to the asleep state than the threshold state is. Furthermore, when offset cycles are to be included in the repeated cycles, in other words, when offset cycles are to be generated, the stimulation pattern may include a frequency of offset cycles with respect to synchronization cycles (for example, one offset cycle with respect to two synchronization cycles, or one offset cycle with respect to three synchronization cycles). Awakening inducing device 100a includes, for example, an operator (not illustrated), such as a keyboard or a touch panel, to be operated by user 500. Control device 120a, for example, obtains information indicating a vibration pattern obtained from user 500 via the operator and determines a vibration pattern based on the obtained information. Alternatively, control device 120a may determine a vibration pattern based on the degree of drowsiness of user 500.

Next, control device 120a performs control of driving vibration devices 110a such that vibration devices 110a vibrate in the vibration pattern determined at step S206 (step S207). With this control, control device 120a, by controlling vibration devices 110a, causes vibration devices 110a to give vibration stimulation to at least one of muscle belly portion 520 or insertion portion 530 of the left leg of user 500. It should be noted that the same steps as step 104 and step 105 in Embodiment 1 are also performed after step S207, although the steps are not shown in the flowchart.

In this example, control device 120a determines at step S205 whether the degree of drowsiness of user 500 exceeds the predetermined degree of drowsiness. Alternatively, control device 120a may determine at step S205 whether the degree of drowsiness of user 500 is equal to or higher than the predetermined degree of drowsiness. For example, in a case where the degree of drowsiness is expressed in a numerical value and the drowsiness of user 500 is defined to be higher as the numerical value is higher, that the degree of drowsiness of user 500 exceeds the predetermined degree of drowsiness means that the numerical value representing the degree of drowsiness of user 500 is higher that the predetermined degree of drowsiness expressed by a predetermined numerical value or that the numerical value representing the degree of drowsiness of user 500 is equal to or higher than the predetermined degree of drowsiness expressed by a predetermined numerical value.

### Advantageous Effects and Others

As described above, awakening inducing device 100a according to Embodiment 2 includes vibration devices 110a and control device 120a. Vibration devices 110a are disposed in seat 301 at respective positions where vibration devices 110a oppose at least one of muscle belly portion 520 or insertion portion 530 of a hamstring of user 500 sitting in seat 301. Control device 120a, by controlling vibration devices 110a, causes vibration devices 110a to give vibration stimulation to at least one of muscle belly portion 520 or insertion portion 530. Awakening inducing device 100a further includes footrest 140a on which user 500 places at least one foot. In this case, footrest 140a is disposed at a position where, when user 500 sitting in seat 301 has placed a foot on footrest 140a, at least one of muscle belly portion 520 or insertion portion 530 is in contact with seat 301. In awakening inducing device 100a, seat 301 is the driver's seat of a vehicle. In this case, footrest 140a is disposed at a position where user 500 sitting in seat 301 places his or her left foot.

In a case where seat 301 is the driver's seat of a vehicle, vibration devices 110a are disposed at respective positions where vibration devices 110a oppose only the left leg of user 500 sitting in seat 301.

If user 500 is startled by vibration stimulation while driving or if vibration stimulation is undesirable and unpleasant to user 500, this may cause user 500 to unintentionally move the leg to which the vibration stimulation has been given. The driving action of stepping on the accelerator or the brake is often carried out with the right foot. Therefore, giving vibration stimulation to the left leg can prevent a situation in which the driving of user 500 is impeded, even if user 500 has unintentionally moved the leg to which the vibration stimulation has been given. As footrest 140a is disposed at the position where the left foot is placed, when user 500 has placed the left foot on footrest 140a, user 500 can be brought into a posture that allows vibration devices 100a to give vibration stimulation appropriately to at least one of muscle belly portion 520 or insertion portion 530 of the left leg.

Awakening inducing system 200a according to Embodiment 2 includes awakening inducing device 100a, seat 301, state detecting sensor 210 that detects state information indicating a state of user 500 sitting in seat 301, and determining device 220 that determines the degree of drowsiness of user 500 based on the state information detected by state detecting sensor 210. In this case, control device 120a controls vibration devices 110a based, for example, on the determination result of determining device 220.

With this configuration, when it is determined that user 500 needs to be awakened based on the degree of drowsiness of user 500, user 500 can be awakened appropriately by being given vibration stimulation to at least one of muscle belly portion 520 or insertion portion 530 of user 500.

### Embodiment 3

Next, an awakening inducing system that includes an awakening inducing device according to Embodiment 3 will be described. The description of the awakening inducing system according to Embodiment 3 centers on the differences from awakening inducing system 200 according to Embodiment 1 or awakening inducing system 200a according to Embodiment 2. In the description of the awakening inducing system according to Embodiment 3, configurations identical to those of awakening inducing system 200 according to Embodiment 1 or awakening inducing system 200a according to Embodiment 2 are given identical reference characters, and their description will be partly simplified or omitted.

### [Configuration]

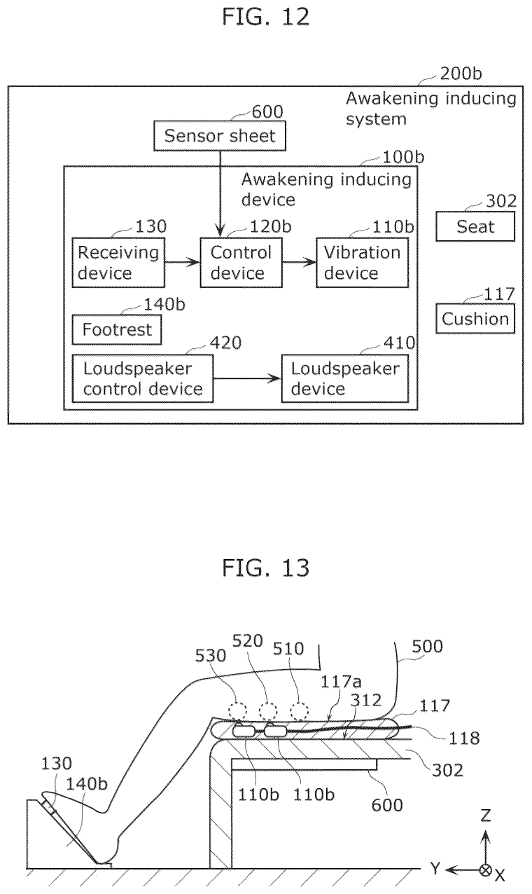

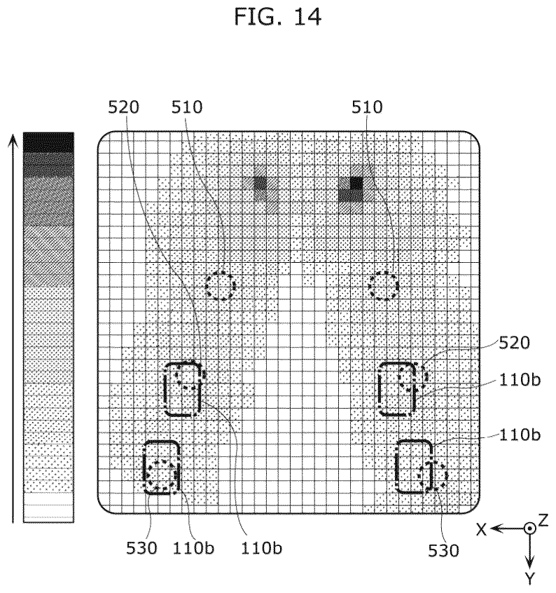

FIG. 12 is a block diagram illustrating a functional configuration of awakening inducing system 200b according to Embodiment 3. FIG. 13 is an illustration for explaining positions on user 500 to which vibration devices 110b according to Embodiment 3 give vibration stimulation. FIG. 14 is a diagram illustrating an example of a pressure distribution across seating surface 312 of seat 302. FIG. 13 schematically illustrates the inside of cushion 117 for description. FIG. 14 shows four vibration devices 110b, whereas FIG. 12 shows only one vibration device 110b for description. FIG. 14 shows a location with a relatively higher pressure by a deeper black and a location with a relatively lower pressure by a lighter black.

Awakening inducing system 200b includes seat 302, cushion 117, sensor sheet 600, and awakening inducing devices 100b.

Awakening inducing device 100b are devices that awaken user 500 by giving vibration stimulation to at least one of muscle belly portion 520 or insertion portion 530 of one or each of the hamstrings of user 500 sitting on seat 302. According to the present embodiment, cushion 117 is disposed on seating surface 312 of seat 302.

Cushion 117 is, for example but not limited to, a cushion placed on seating surface 312 of seat 302 or a sheet that covers seating surface 312. According to the present embodiment, vibration devices 110b are disposed inside cushion 117. User 500 sits on seat 302 with cushion 117 interposed therebetween. Specifically, user 500 sits on seating surface 117a of cushion 117 disposed on seating surface 312 of seat 302. It suffices that vibration devices 110b be provided on seat 302 at at least one of a position where vibration devices 110b oppose muscle belly portion 520 of a hamstring of user 500 sitting on seat 302 or a position where vibration devices 110b oppose insertion portion 530 of a hamstring of user 500 sitting on seat 302, and vibration devices 110b may be provided inside seat 302, under seat 302, or on top of or above seat 302. For example, as illustrated in FIG. 13, vibration devices 110b may be disposed at respective positions where vibration devices 110b can give vibration stimulation to user 500 via seating surface 312 of seat 302 on which user 500 sits (according to the present embodiment, via seating surface 117a of cushion 117 placed on seating surface 312 of seat 302).

In this example, a recess portion may be formed in cushion 117 by reducing the thickness of a part of a urethane sponge or the like that cushion 117 includes for cushioning effect, and vibration devices 110b may be disposed in that recess portion. This configuration can reduce the likelihood that user 500 feels discomfort due to the thickness of vibration devices 110b.

For example, eccentric motor 111 (see FIG. 5) of each vibration device 110b is electrically connected to a power source (not illustrated), such as a battery, via power source cord 118 or the like and is driven as the power from the power source is supplied to eccentric motor 111.

In this example, power source cord 118 preferably extends behind (i.e., on the side of the backrest of seat 302) or on the side (e.g., the direction in which an armrest or the like of seat 302 is disposed) of user 500 so that power source cord 118 does not interfere with sitting user 500.

Sensor sheet 600 is a sensor for measuring the distribution of pressure exerted on seating surface 312 of seat 302 by user 500 sitting on seat 302. Sensor sheet 600 is disposed, for example, on the side of seat 302 opposite to the side where seating surface 312 is located. Control device 120b obtains pressure distribution information from sensor sheet 600, and the pressure distribution information is information that indicates the pressure distribution across seating surface 312.

Sensor sheet 600 obtains pressure distribution information that indicates a pressure distribution such as the one illustrated in FIG. 14, for example. For example, on seating surface 312, each gluteus maximus of user 500 is located at a position where the pressure is highest. The pressure decreases gradually along a relatively large distance from the position where the pressure is highest, and this gradual decrease occurs in one direction (the Y-axis direction according to the present embodiment) as compared to other directions. Each hamstring extends in this one direction from the position where the pressure is highest. Therefore, control device 120b can estimate the position of each hamstring of user 500 based on the pressure distribution information and can thus estimate the position of origin portion 510, muscle belly portion 520, and insertion portion 530.

In this example, it suffices that sensor sheet 600 be capable of measuring the pressure distribution, and sensor sheet 600 may detect the pressure based on the change in the resistance value or based on the change in the capacitance.

Awakening inducing device 100b includes vibration devices 110b, control device 120b, receiving device 130, and footrest 140b. Control device 120b is connected to each vibration device 110b and receiving device 130 such that these connected devices can transmit or receive signals therebetween via a wired circuit or a wireless circuit.

Vibration devices 110b are each a device that gives vibration stimulation to at least one of muscle belly portion 520 or insertion portion 530.

According to the present embodiment, vibration devices 110b are disposed inside cushion 117 so as to give vibration stimulation to seating surface 117a of cushion 117. For example, vibration devices 110b are disposed in cushion 117 such that vibration devices 110b do not move inside cushion 117 and can vibrate inside cushion 117. Vibration devices 110b are provided on seat 302 for the user at at least one of positions where vibration devices 110b oppose muscle belly portions 520 of the hamstrings of the legs of user 500 sitting on seat 302 or positions where vibration devices 110b oppose insertion portions 530 of the hamstrings of the legs of user 500 sitting on seat 302.

Vibration devices 110b each give vibration stimulation to at least one of muscle belly portion 520 or insertion portion 530 of one or each of the hamstrings of user 500 sitting on seat 302 via cushion 117 by, for example, vibrating in a vibration direction that is parallel to the direction orthogonal to the extending direction of the hamstrings.

Control device 120b is a device that, by controlling the driving of vibration devices 110b, causes vibration devices 110b to give vibration stimulation to at least one of muscle belly portion 520 or insertion portion 530 of user 500 via seating surface 117a of cushion 117 placed on seating surface 312 of seat 302.

Control device 120b includes, for example, a communication interface for transmitting or receiving a signal to or from each vibration device 110b, receiving device 130, or sensor sheet 600, a memory such as a flash memory or an HDD storing a control program, and a CPU that executes the control program.

Footrest 140b is a support on which user 500 places at least one foot. According to the present embodiment, footrest 140b is disposed at a position where user 500 places his or her left foot. Specifically, footrest 140b is inclined in part where footrest 140b receives a foot or the feet of user 500. There is no particular limitation on the shape of footrest 140b.

### [Processing Procedure]

Next, processing procedures of awakening inducing system 200b according to Embodiment 3 will be described.

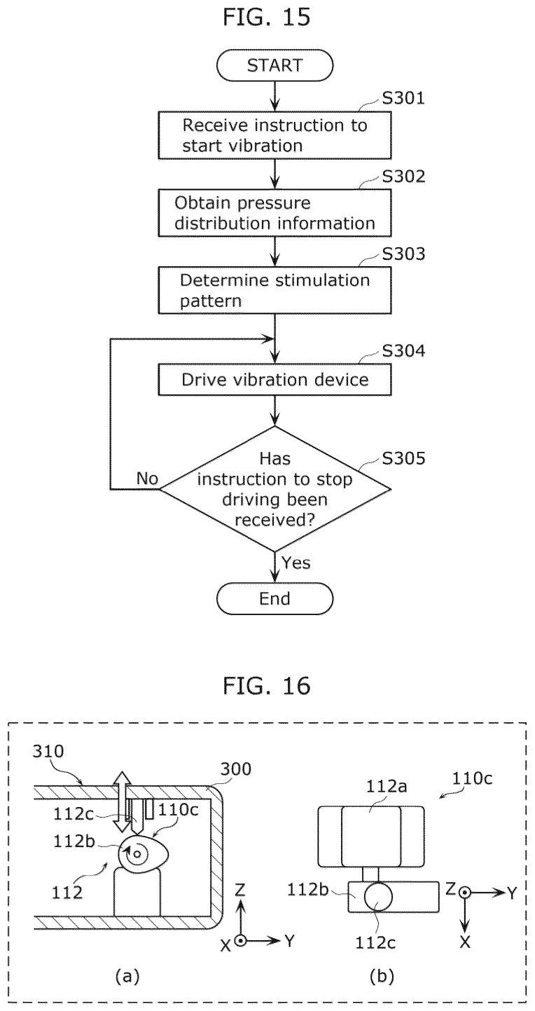

FIG. 15 is a flowchart for explaining a process executed by awakening inducing system 200b according to Embodiment 3.

First, receiving device 130 receives an instruction from user 500 that causes vibration devices 110b to start being driven (step S301). For example, in a case where receiving device 130 is a push button, user 500 has depressed the push button.

Next, control device 120b, for example, obtains pressure distribution information from sensor sheet 600 (step S302).

Next, control device 120b determines a stimulation pattern of vibration stimulation to be given to user 500 (step S303). Determining this stimulation pattern includes, for example, selecting vibration device 110b to select the position on user 500 to which the vibration stimulation is to be given. For example, control device 120b selects vibration device 110b to be caused to vibrate from a plurality of vibration devices 110b based on the pressure distribution information obtained from sensor sheet 600 at step S302. For example, awakening inducing device 100b includes a plurality of vibration devices 110b each disposed at a different position. Based on the pressure distribution information obtained from sensor sheet 600 at step S302, control device 120b selects vibration device 110b that is disposed at a position where selected vibration device 110b can give vibration stimulation to at least one of muscle belly portion 520 or insertion portion 530 (e.g., vibration device 110b located at a position where vibration device 110b opposes at least one of muscle belly portion 520 or insertion portion 530). Information that indicates the position of each vibration device 110b may be stored in advance in a memory of control device 120b.

Next, control device 120b performs control of driving vibration devices 110b such that vibration devices 110b vibrate in the vibration pattern determined at step S303 (step S304). With this control, control device 120b, by controlling vibration devices 110b, causes vibration devices 110b to give vibration stimulation to at least one of muscle belly portion 520 or insertion portion 530. It should be noted that the steps same as step S104 and S105 are performed after step 304, although the steps are not illustrated in the flowchart.

Next, control device 120b determines whether receiving device 130 has received an instruction from user 500 that causes vibration devices 110b to stop being driven (step S305).

If control device 120b determines that receiving device 130 has received an instruction from user 500 that causes vibration devices 110b to stop being driven (Yes at step S305), control device 120b causes vibration devices 110b to stop being driven and terminates the process.

Meanwhile, if control device 120b determines that receiving device 130 has not received any instruction from user 500 that causes vibration devices 110b to stop being driven (No at step S305), control device 120b returns the process to step S304.

As described above, for example, control device 120b selects and drives vibration device 110b, among a plurality of vibration devices 110b, that can give vibration stimulation to at least one of muscle belly portion 520 or insertion portion 530 of user 500 based on the pressure distribution information obtained from sensor sheet 600. For example, the awakening inducing device may include a mover for changing the position of each vibration device 110b. The mover includes, for example but not limited to, a motor and a guide for moving vibration device 110b. In this case, the control device may move vibration device 110b and then drive moved vibration device 110b (cause moved vibration device 110b to vibrate) based on the pressure distribution information obtained from sensor sheet 600. This configuration allows the awakening inducing device to move vibration device 110b to an appropriate position and then give vibration stimulation to at least one of muscle belly portion 520 or insertion portion 530 of user 500. The position on user 500 to which vibration device 110b gives vibration stimulation is determined by the relative positional relationship between seating surface 312 (seating surface 117a according to the present embodiment) and vibration device 110b if the position where user 500 sits remains Therefore, for example, awakening inducing device unchanged. 100b may include a mover, such as a motor, that can change the position of seating surface 312 (or seating surface 117a) relative to vibration device 110b. Control device 120b may change the position of seating surface 312 (or seating surface 117a) by controlling the mover based on the pressure distribution information and change the position on user 500 to which vibration stimulation is given.

Awakening inducing device 100b may further include a presenter that includes, for example but not limited to, a display for displaying an image or an audio device, such as an amplifier or a speaker, for providing an audible output. Control device 120b may determine whether the position on user 500 to which vibration stimulation is to be given is appropriate based on the pressure distribution information and prompt user 500 to sit at an appropriate position by controlling the presenter based on the determination result. For example, in a case where the presenter is an audio device, control device 120b may prompt user 500 to sit at an appropriate position by causing the audio device to provide an audible output stating, for example, “please sit deeper in the seat”. Alternatively, in a case where the presenter is a display, control device 120b may prompt user 500 to sit at an appropriate position by displaying an image of an appropriate posture on the display.

### [Variations]

### [Variations of Variation Device]

Vibration devices 110 each include eccentric motor 111 in the foregoing description, but the configuration for causing the vibration devices to vibrate is not limited to eccentric motor 111.

FIG. 16 is a diagram illustrating a first variation of the vibration device. Specifically, (a) in FIG. 16 is a side view illustrating the first variation of the vibration device, and (b) in FIG. 16 is a top view illustrating the first variation of the vibration device. Part (a) of FIG. 16 shows a part of the drawing in section for description.

Vibration device 110c illustrated in FIG. 16 includes rotary motor 112 with a cam mechanism, in place of eccentric motor 111.

Rotary motor 112 with a cam mechanism includes motor 112a, cam 112b, and contact element 112c.

Motor 112a is a motor that rotates cam 112b in response to the power supplied to motor 112a.

Cam 112b is a non-circular structure. Cam 112b is disposed in contact with contact element 112c.

Contact element 112c is a pin that is moved vertically by cam 112b. Vibration device 110c gives vibration stimulation to user 500 sitting on seating surface 310 by use of contact element 112c.

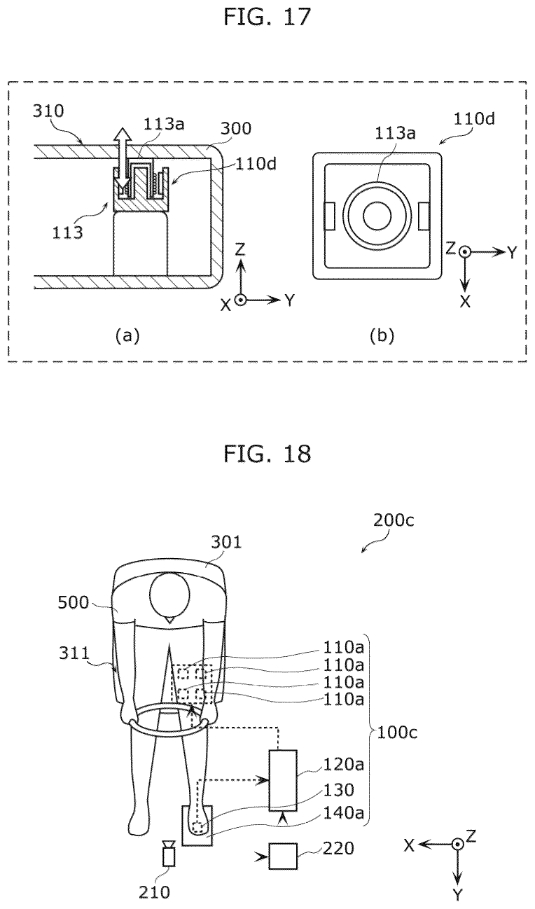

FIG. 17 is a diagram illustrating a second variation of the vibration device. Specifically, (a) in FIG. 17 is a side view illustrating the second variation of the vibration device, and (b) in FIG. 17 is a top view illustrating the second variation of the vibration device. Part (a) of FIG. 17 shows a part of the drawing in section for description.

Vibration device 110d illustrated in FIG. 17 includes voice coil motor 113.

Voice coil motor 113 causes movable element 113a to vibrate vertically by generating a magnetic field in response to the power supplied to voice coil motor 113. Vibration device 110d gives vibration stimulation to user 500 sitting on seating surface 310 by use of movable element 113a.

As described above, for example, the vibration device includes eccentric motor 111, rotary motor 112 with a cam mechanism, or voice coil motor 113. The vibration device is disposed in or on seat 300 such that the vibration direction coincides with the vertical direction, for example.

When the vibration device includes eccentric motor 111, rotary motor 112 with a cam mechanism, or voice coil motor 113, the vibration device can be made to vibrate only in one direction with a simple configuration. Therefore, such a configuration makes it possible to cause the vibration device to vibrate in the vertical direction with a simple configuration only by disposing the vibration device appropriately. A vertically downward force is constantly applied to the body of user 500 sitting on seat 301 due to the gravitational force. When vibration stimulation in the vertical direction is given to user 500, the vibration direction coincides with the direction of the force that acts on the body of user 500. This configuration makes it possible to give vibration stimulation efficiently to at least one of muscle belly portion 520 or insertion portion 530 of user 500 while the body of user 500 remains in contact with seat 300 with an appropriate pressure. Therefore, when the vibration device includes eccentric motor 111, rotary motor 112 with a cam mechanism, or voice coil motor 113 and when the vibration direction of the vibration device coincides with the vertical direction, control device 120 can cause the vibration device to give vibration stimulation to user 500 more effectively by causing the vibration device to vibrate in the vertical direction with a simple configuration.

### [Variation of Arrangement of Variation Devices]

FIG. 18 is a diagram illustrating a variation in how vibration devices 110a are disposed. Awakening inducing system 200c illustrated in FIG. 18 is, for example, a variation of awakening inducing system 200a illustrated in FIG. 8 and differs from awakening inducing system 200a in terms of the number of vibration devices 110a.

Four vibration devices 110a included in awakening inducing device 100c illustrated in FIG. 18 are disposed at respective positions where vibration devices 110a oppose at least one of muscle belly portion 520 or insertion portion 530 of the left leg of user 500. In this example, four vibration devices 110a are disposed in seat 301 such that vibration devices 110a are located at positions that mutually differ in the front-back direction and the right-left direction as viewed from user 500 sitting in seat 301, for example.

For example, each vibration device 110a gives vibration stimulation only to the left leg that user 500 does not use for driving (i.e., the left leg that user 500 does not use to operate the accelerator or the brake). In this example, each vibration device 110a may be used for purposes other than the purpose of awakening user 500. For example, vibration devices 110a may be used as an attention function with the purpose of supporting user 500 in driving, and the attention function is a function of prompting user 500 to pay attention. Alternatively, vibration devices 110a may be used as an alert function for the purpose of supporting user 500 in driving, and the alert function is a function of alerting user 500 to an impending danger, for example.

The attention function is a function of, for example, presenting the direction in which user 500, the driver, is to travel in the form of vibration stimulation, by cooperating with a car navigation system (not illustrated). For example, two vibration devices 110a are disposed so as to oppose the right and left sides of insertion portion 530 of the hamstring of the left leg. When control device 120a, serving for example as a car navigation system, presents user 500 with information indicating that user 500 should turn right at the next intersection, control device 120a drives vibration device 110a located so as to oppose the right side of insertion portion 530 of the hamstring of the left leg before user 500 enters the intersection. This configuration enables user 500 to recognize, via a tactile sense, that user 500 should turn right at the next intersection.

Alternatively, control device 120a can present a warning, instead of indicating the direction of travel, by causing two vibration devices 110a disposed on the right and left sides to vibrate in an alternating manner with some time difference or by causing two vibration devices 110a disposed on the right and left sides to vibrate simultaneously. For example, awakening inducing system 200c may include a camera (not illustrated) for capturing an image of the surroundings of the vehicle driven by user 500. Control device 120a may, for example, obtain an image from the camera, determine the presence of any obstruction in the surroundings of the vehicle by analyzing the image, and drive vibration devices 110a to present a warning based on the determination result.

As described above, awakening inducing device 100c includes vibration devices 110a and control device 120a. Vibration devices 110a are provided in or on seating surface 311 of seat 301 at respective positions where vibration devices 110a oppose a gluteus maximus or a thigh portion of user 500 when user 500 serving as the driver sits in seat 301 in the vehicle and give vibration stimulation to at least one of the gluteus maximus or the thigh portion of user 500. Control device 120a controls the driving of vibration devices 110a. Vibration devices 110a are provided so as to correspond to the left leg of user 500. Control device 120a, for example, performs at least one of presenting user 500 with the traveling direction or presenting user 500 with a warning by controlling vibration devices 110a.

With this configuration, control device 120a can perform at least one of presenting user 500 with the traveling direction or presenting user 500 with a warning by controlling vibration devices 110a without interfering with the driving of user 500.

### Other Embodiments

Thus far, awakening inducing devices and awakening inducing systems according to the present disclosure have been described based on some embodiments and variations, but the present disclosure is not limited to the embodiments and the variations described above. For example, an embodiment obtained by making various modifications that a person skilled in the art can conceive of to the foregoing embodiments and variations or an embodiment achieved by combining, as desired, the constituent elements and the functions in the foregoing embodiments within the scope that does not depart from the spirit of the present disclosure is also encompassed by the present disclosure.

For example, according to the foregoing embodiments, an awakening inducing system includes a state detecting sensor, a determining device, and so on. Alternatively, an awakening inducing device may include a state detecting sensor, a determining device, and so on. In this manner, the constituent elements of the awakening inducing devices or the awakening inducing systems described above are merely examples and do not limit the present disclosure.

For example, constituent elements of a processor in a control device, a determining device, or the like may be constituted by one or more electronic circuits. The one or more electronic circuits may each be a general purpose circuit or a dedicated circuit. The one or more electronic circuits may include a semiconductor device, an integrated circuit (IC), or a large scale integration (LSI) circuit, for example. An IC or an LSI circuit may be integrated into a single chip or into a plurality of chips. Although the term used herein is IC or LSI circuit, a circuit may also be called a system LSI circuit, a very large scale integration (VLSI) circuit, or an ultra large scale integration (ULSI) circuit depending on the degree of integration. A field programmable gate array (FPGA) that can be programmed after manufacturing an LSI circuit can also be used for the same purpose.

General or specific aspects of the present disclosure may be implemented in the form of a system, a device, a method, an integrated circuit, or a computer program. Alternatively, the general and specific aspects may be implemented in the form of a computer readable non-transitory recording medium, such as an optical disc, an HDD, or a semiconductor memory, storing the computer program. Furthermore, the general or specific aspects may be implemented through a desired combination of a system, a device, a method, an integrated circuit, a computer program, and a recording medium.

Further Information about Technical Background to this Application

The disclosures of the following patent applications including specification, drawings, and claims are incorporated herein by reference in their entirety: Japanese Patent Application No. 2022-147602 filed on Sep. 16, 2022, and PCT International Application No. PCT/JP2023/022586 filed on Jun. 19, 2023.

### INDUSTRIAL APPLICABILITY

The present disclosure can be used in, for example but not limited to, a device that awakens a user such as a driver driving a mobile body while sitting in a seat.

1. An awakening inducing device comprising:
a first awakener that induces awakening of a user by periodically repeating a first stimulation period in which a stimulus of a first type is applied to the user and a first suspension period in which no stimulus is applied to the user;
a second awakener that induces awakening of the user by periodically repeating a second stimulation period in which a stimulus of a second type is applied to the user and a second suspension period in which no stimulus is applied to the user; and
a controller that causes the first awakener and the second awakener to have a synchronization cycle and an offset cycle, the synchronization cycle being a cycle in which a start timing of the first stimulation period and a start timing of the second stimulation period are synchronized with each other, the offset cycle being a cycle in which the start timing of the first stimulation period and the start timing of the second stimulation period are offset from each other.

2. The awakening inducing device according to claim 1, wherein
the stimulus of the first type is sound, and
the stimulus of the second type is vibration.

3. The awakening inducing device according to claim 1, wherein
the stimulus of the first type is light, and
the stimulus of the second type is sound.

4. The awakening inducing device according to claim 1, wherein
the stimulus of the first type is light, and
the stimulus of the second type is vibration.

5. The awakening inducing device according to claim 1, wherein
the stimulus of the first type is vibration, and
the stimulus of the second type is vibration different from the vibration of the stimulus of the first type.

6. The awakening inducing device according to claim 2, wherein
a length of the offset between the start timing of the first stimulation period and the start timing of the second stimulation period is at least 40 milliseconds (ms) and at most half of the second suspension period.

7. The awakening inducing device according to claim 1, wherein
the controller causes the first awakener and the second awakener to repeat a cycle pattern that includes the offset cycle once and the synchronization cycle one or more times in a predetermined order.

8. The awakening inducing device according to claim 1, wherein
the controller causes the first awakener and the second awakener to repeat a cycle pattern that includes the offset cycle once and the synchronization cycle one or more times, and
the offset cycle and the synchronization cycle are in a random order in the cycle pattern.

9. The awakening inducing device according to claim 1, further comprising:
a drowsiness estimator that estimates a state of drowsiness of the user, wherein
the controller causes the first awakener and the second awakener to have the synchronization cycle only, when the state of drowsiness estimated is closer to an awake state than a threshold state is, and
the controller causes the first awakener and the second awakener to have both the synchronization cycle and the offset cycle, when the state of drowsiness estimated is closer to an asleep state than the threshold state is.

10. The awakening inducing device according to claim 1, further comprising:
a drowsiness estimator that estimates a state of drowsiness of the user, wherein
the controller changes, based on the state of drowsiness estimated, a stimulation condition for stimulation applied to the user by at least one of the first awakener or the second awakener.

11. The awakening inducing device according to claim 10, wherein
the drowsiness estimator estimates a level of drowsiness of the user as the state of drowsiness, and
the controller changes the stimulation condition for stimulation applied to the user by at least one of the first awakener or the second awakener, when the level of drowsiness estimated exceeds a predetermined level.

12. The awakening inducing device according to claim 10, wherein
the drowsiness estimator estimates a temporal change of a level of drowsiness of the user as the state of drowsiness, and
the controller changes the stimulation condition for stimulation applied to the user by at least one of the first awakener or the second awakener, when a degree of the temporal change estimated is equal to or greater than a predetermined degree.

13. The awakening inducing device according to claim 10, wherein
the drowsiness estimator estimates a future level of drowsiness of the user as the state of drowsiness, and
the controller changes the stimulation condition for stimulation applied to the user by at least one of the first awakener or the second awakener, when the future level of drowsiness estimated exceeds a predetermined level.

14. The awakening inducing device according to claim 1, wherein
the controller randomly changes a stimulation condition for stimulation applied to the user by at least one of the first awakener or the second awakener.

15. The awakening inducing device according to claim 10, wherein
the controller changes, as the stimulation condition, a frequency of the offset cycle with respect to the synchronization cycle.

16. The awakening inducing device according to claim 10, wherein
the controller changes, as the stimulation condition, a length of the offset between the start timing of the first stimulation period and the start timing of the second stimulation period in the offset cycle.

17. The awakening inducing device according to claim 10, wherein
at least one of the stimulus of the first type or the stimulus of the second type is vibration, and
the controller changes, as the stimulation condition, a vibration frequency of the vibration applied by at least one of the first awakener or the second awakener.

18. The awakening inducing device according to claim 10, wherein
the controller changes, as the stimulation condition, a length of at least one of the stimulus of the first type or the stimulus of the second type.

19. The awakening inducing device according to claim 10, wherein
at least one of the stimulus of the first type or the stimulus of the second type is vibration,
at least one of the first awakener or the second awakener applies the vibration to two or more different portions on the user, and
the controller changes, as the stimulation condition, the two or more different portions on which the vibration is applied by the at least one of the first awakener or the second awakener to two or more other portions on the user.

20. The awakening inducing device according to claim 10, wherein
the controller changes, as the stimulation condition, an intensity of at least one of the stimulus of the first type or the stimulus of the second type.

21. An awakening inducing method comprising:
inducing awakening of a user by periodically repeating a first stimulation period in which a stimulus of a first type is applied to the user and a first suspension period in which no stimulus is applied to the user;
inducing awakening of the user by periodically repeating a second stimulation period in which a stimulus of a second type is applied to the user and a second suspension period in which no stimulus is applied to the user; and
causing the stimulus of the first type and the stimulus of the second type applied to the user to have a synchronization cycle and an offset cycle, the synchronization cycle being a cycle in which a start timing of the first stimulation period and a start timing of the second stimulation period are synchronized with each other, the offset cycle being a cycle in which the start timing of the first stimulation period and the start timing of the second stimulation period are offset from each other.

22. A non-transitory computer-readable recording medium for use in a computer, the recording medium having recorded thereon a program for causing the computer to execute:
the awakening inducing method according to claim 21.
